(19) United States
(12) Patent Application Publication
(10) Pub. No.: US 2025/0221967 A1
(43) Pub. Date: Jul. 10, 2025

(54) COMPOSITIONS AND METHODS FOR THE TREATMENT OF CANCER AND OTHER PROLIFERATIVE DISEASES

(71) Applicant: THE WISTAR INSTITUTE OF ANATOMY AND BIOLOGY, Philadelphia, PA (US)

(72) Inventor: Luis J. MONTANER, Philadelphia, PA (US)

(73) Assignee: THE WISTAR INSTITUTE OF ANATOMY AND BIOLOGY, (US)

(21) Appl. No.: 18/850,798

(22) PCT Filed: Mar. 14, 2023
(86) PCT No.: PCT/US2023/064295

§ 371 Date: Sep. 25, 2024

Related U.S. Application Data
(60) Provisional application No. 63/328,726, filed on Apr. 7, 2022.

(51) Int. Cl. A61K 31/433; A61K 31/17; A61K 31/519; A61K 45/06; A61P 35/00; C07C 275/50; C07D 285/08; C07D 487/04; C07D 493/10

(52) U.S. Cl. CPC A61K 31/433; A61K 31/17; A61K 31/519; A61K 45/06; A61P 35/00; C07C 275/50; C07D 285/08; C07D 487/04; C07D 493/10

(57) ABSTRACT

The present disclosure relates generally to Retinoblastoma (Rb1) protein targeting 1, 2, 4-thiadiazolidin-3, 5-dione compounds, wherein the Rb1 protein is a known tumor suppressor protein, and methods of use thereof. In some aspects, the present disclosure relates to using such Rb1 protein targeting compounds to treat cancer or other hyperproliferative diseases, such as pancreatic cancer.

This application claims the benefit of priority to U.S. Provisional Application No. 63/328,726, filed on Apr. 7, 2022, the entire contents of which are hereby incorporated by reference.

### BACKGROUND

### I. Field

The present disclosure relates generally to composition and methods for treating cancers and other hyperproliferative diseases, such as pancreatic cancers.

### II. Description of Related Art

Pancreatic cancer arises when cells in the pancreas begin to multiply out of control and form a mass. These cancerous cells have the ability to invade other parts of the body. A number of types of pancreatic cancer are known, the most common being pancreatic adenocarcinoma (PA) which accounts for about 90% of cases. These adenocarcinomas start within the part of the pancreas that makes digestive enzymes. Several other types of cancer, representing the majority of the non-adenocarcinomas, can also arise from these cells. About 1-2% of cases of pancreatic cancer are neuroendocrine tumors, which arise from the hormone-producing cells of the pancreas. These are generally less aggressive than PA.

In 2015, pancreatic cancers of all types resulted in 411,600 deaths globally. Pancreatic cancer is the fifth-most-common cause of death from cancer in the United Kingdom, and the third most-common in the United States. The disease occurs most often in the developed world, where about 70% of the new cases in 2012 originated. PA typically has a very poor prognosis; after diagnosis, 25% of people survive one year and 5% live for five years. For cancers diagnosed early, the five-year survival rate rises to about 20%. Neuroendocrine cancers have better outcomes; at five years from diagnosis, 65% of those diagnosed are living, though survival considerably varies depending on the type of tumor.

Pancreatic ductal adenocarcinoma (PDA), a specific form of PA, remains among the cancers with the poorest prognosis. Currently, no effective treatment available for PDA. Immunotherapy that has been shown effective for several cancer types is not successful for PDA treatment overall. The possible reasons of this PDA immunotherapy resistance include lack of neoantigen mutations, low T cell responses and overall immunosuppression state in the tumor microenvironment mediated by myeloid cells and tumor stroma. Recent studies suggest that targeting tumor microenvironment can overcome this immunotherapy resistance and significantly improve the disease outcome.

Similarly, Acute myeloid leukemia (AML) is the most common form of leukemia in adults. In 2017, 120,000 new AML cases of were reported worldwide, with an expectation to reach double the number by 2040. Presently, with an 8.5 months of median survival rate and approximately 60% recurrence rate following chemotherapy treatment, leads to the opportunity to improve overall AML cancer therapy. The Retinoblastoma (Rb1) protein can control canonical cell cycle progression and be a determinant to apoptosis depending on its phosphorylation state, binding partners, and localization (specifically mitochondrial Rb1). Therefore, new compounds that modulate Rb1 could be of use in AML and PDA treatment.

### SUMMARY

In accordance with the present disclosure, there is provided methods of treating cancer comprising administering to a patient in need thereof a therapeutically effective amount of a compound of the formula:

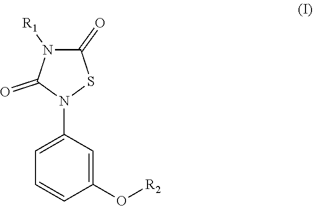

wherein:
   R₁ is hydrogen, alkyl_{(C≤12)}, substituted alkyl_{(C≤12)}, aralkyl_{(C≤12)}, or substituted aralkyl_{(C≤12)}; and
   R₂ is an organic moiety; or
a compound of the formula:

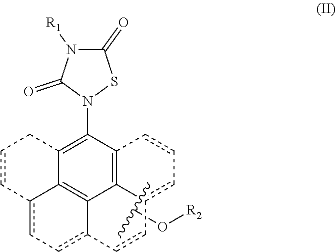

wherein:
   the dashed lines represent one or more fused aromatic ring systems;
   R₁ is hydrogen, alkyl_{(C≤12)}, substituted alkyl_{(C≤12)}, aralkyl_{(C≤12)}, or substituted aralkyl_{(C≤12)}; and
   R₂ is an organic moiety; or
a compound of the formula:

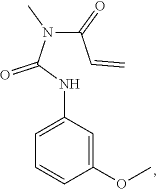 [SMILES: C=CC(=O)N(C)C(=O)Nc1cccc(OC)c1]

or a pharmaceutically acceptable salt or tautomer thereof.

In some embodiments, R₁ is alkyl_{(C≤12)} or substituted alkyl_{(C≤12)}. In some embodiments, R₁ is alkyl_{(C≤12)} such as methyl. In other embodiments, R₁ is substituted alkyl_{(C≤12)}. In other embodiments, R₁ is aralkyl_{(C≤12)} or substituted aralkyl_{(C≤12)}. In some embodiments, R₁ is aralkyl_{(C≤12)} such as benzyl.

In some embodiments, R₂ is an organic moiety selected from a linking group selected from —C(O)—, —C(S)—, —C(O)O—, —C(S)O—, —OC(O)—, —OC(S)—, —C(O)NR_{b}—, —C(S)NR_{b}—, —NR_{b}C(O)—, —NR_{b}C(S)—, —OC(O)O—, —OC(S)O—, —NR_{b}C(O)NR_{b}—, —NR_{b}C(S)NR_{b}—, —S(O)ₓ—, —OS(O)ₓO—, —OS(O)ₓ—, —S(O)ₓO—, and a combination thereof; wherein: x is 0, 1, or 2; and R_{b} is hydrogen, alkyl_{(C≤6)}, or substituted alkyl_{(C≤6)}, a small molecule targeting agent, hydrogen, amino, cyano, halo, hydroxy, nitro, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, alkylamino_{(C≤12)}, dialkylamino_{(C≤12)}, cycloalkylamino_{(C≤12)}, cycloalkyl(alkyl)amino_{(C≤12)}, dicycloalkylamino_{(C≤12)}, alkoxy_{(C≤12)}, aryloxy_{(C≤12)}, acyloxy_{(C≤12)}, acyl_{(C≤12)}, amido_{(C≤12)}, alkylsulfonyl_{(C≤12)}, arylsulfonyl_{(C≤12)}, or a substituted version thereof, or a combination thereof.

In some embodiments, R₂ is alkyl_{(C≤12)} or substituted alkyl_{(C≤12)}. In some embodiments, R₂ is alkyl_{(C≤12)} such as methyl. In other embodiments, R₂ is substituted alkyl_{(C≤12)} such as aminopropyl. In some embodiments, R₂ is 3-aminopropyl. In other embodiments, R₂ is a combination of alkyl_{(C≤12)} or substituted alkyl_{(C≤12)} and amido_{(C≤12)} or substituted amido_{(C≤12)}. In some embodiments, R₂ is a combination of alkyl_{(C≤12)} and amido_{(C≤12)}. In some embodiments, the amido_{(C≤12)} of R₂ is —C(O)Ph. In some embodiments, the alkyl_{(C≤12)} of R₂ is ethylene or propylene. In some embodiments, the alkyl_{(C≤12)} of R₂ is propylene.

In other embodiments, R₂ is a combination of an alkyl_{(C≤12)} or substituted alkyl_{(C≤12)}, a linking group, and an imaging agent. In some embodiments, the alkyl_{(C≤12)} of R₂ is ethylene or propylene. In some embodiments, the linking group of R₂ is —C(O)—, —C(S)—, —C(O)O—, —C(S)O—, —OC(O)—, —OC(S)—, —C(O)NR_{b}—, —C(S)NR_{b}—, —NR_{b}C(O)—, —NR_{b}C(S)—, —OC(O)O—, —OC(S)O—, —NR_{b}C(O)NR_{b}—, —NR_{b}C(S)NR_{b}—, —S(O)ₓ—, —OS(O)ₓO—, —OS(O)ₓ—, —S(O)ₓO—, and a combination thereof, wherein: x is 0, 1, or 2; and R_{b} is hydrogen, alkyl_{(C≤6)}, or substituted alkyl_{(C≤6)}. In some embodiments, the linking group of R₂ is —OC(S)O— or —NR_{b}C(S)NR_{b}—. In some embodiments, the linking group of R₂ is —NR_{b}C(S)NR_{b}—. In some embodiments, the imaging agent is a fluorophore such as a fluorescein.

In other embodiments, R₂ is a combination of one or more alkyl_{(C≤12)} or substituted alkyl_{(C≤12)}, a targeting group, an aryl_{(C≤12)} or substituted aryl_{(C≤12)}, and an amino acid. In some embodiments, the alkyl_{(C≤12)} of R₂ is ethylene or propylene. In some embodiments, the targeting group of R₂ is a nucleotide or nucleotide analog. In some embodiments, the aryl_{(C≤12)} of R₂ is a benzenediyl.

In other embodiments, the compound is of formula II. In some embodiments, the compound comprises two aryl rings. In some embodiments, the compound is further defined as:

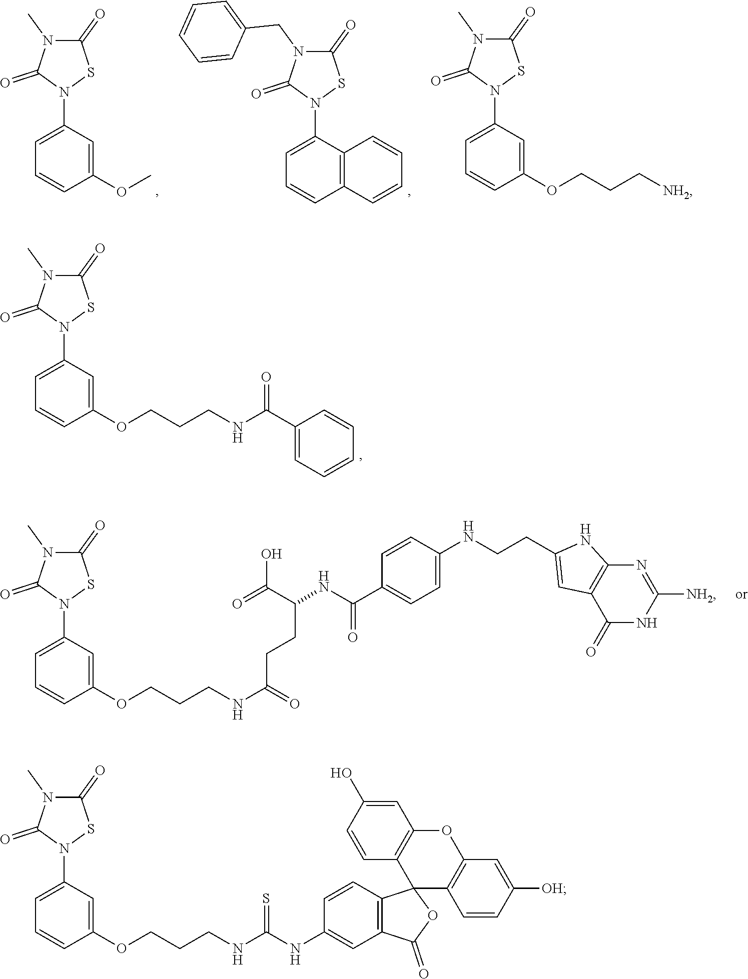 [SMILES: COc1cccc(-n2sc(=O)n(C)c2=O)c1 ; O=c1sn(-c2cccc3ccccc23)c(=O)n1Cc1ccccc1 ; Cn1c(=O)sn(-c2cccc(OCCCN)c2)c1=O ; Cn1c(=O)sn(-c2cccc(OCCCNC(=O)c3ccccc3)c2)c1=O ; Cn1c(=O)sn(-c2cccc(OCCCNC(=O)CC[C@@H](NC(=O)c3ccc(NCCc4cc5c(=O)[nH]c(N)nc5[nH]4)cc3)C(=O)O)c2)c1=O ; Cn1c(=O)sn(-c2cccc(OCCCNC(=S)Nc3ccc4c(c3)C(=O)OC43c4ccc(O)cc4Oc4cc(O)ccc43)c2)c1=O]

or a pharmaceutically acceptable salt thereof.

In some embodiments, the cancer is a solid tumor cancer. In some embodiments, the cancer is a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is a cancer of the pancreas. In some embodiments, the cancer is an adenocarcinoma of the pancreas such as a pancreatic ductal adenocarcinoma. In other embodiments, the cancer is a leukemia such as acute myeloid leukemia.

In some embodiments, the methods comprise modulating the myeloid cells of the cancer. In some embodiments, the cancer is an operable cancer. In other embodiments, the cancer is inoperable. In some embodiments, the inoperable cancer becomes operable after treatment with the compound. In some embodiments, the cancer is a drug resistant cancer.

In some embodiments, the cancer recurrent and/or metastatic.

In some embodiments, the methods comprise injecting the compound directly into the tumor. In some embodiments, the compound is formulated as an injectable solution. In some embodiments, the methods comprise administering the compound systemically. In some embodiments, the compound is formulated for oral or intravenous administration. In some embodiments, the methods further comprise alleviating pain. In some embodiments, the methods further comprise administering a second therapeutic regimen to said patient. In some embodiments, the second therapeutic regimen is surgery, radiotherapy, immunotherapy, genetic therapy, or a second chemotherapeutic compound. In some embodiments, the second therapeutic regimen comprises gemcitabine and/or paclitaxel. In some embodiments, the methods further comprise at least a second, third, fourth, fifth, sixth, seventh, eighth, ninth or tenth administration of said compound.

In still yet another aspect, the present disclosure provides methods of reducing the size of a tumor comprising administering to a patient in need thereof a therapeutically effective amount of a compound of the formula:

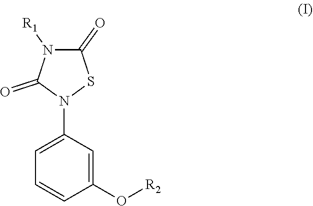

wherein:
   R₁ is hydrogen, alkyl_{(C≤12)}, substituted alkyl_{(C≤12)}, aralkyl_{(C≤12)}, or substituted aralkyl_{(C≤12)}; and
   R₂ is an organic moiety;
or a pharmaceutically acceptable salt or tautomer thereof.

In still yet another aspect, the present disclosure provides compounds of the formula:

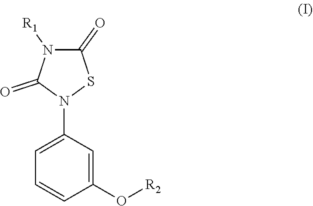

wherein:
   R₁ is hydrogen, alkyl_{(C≤12)}, substituted alkyl_{(C≤12)}, aralkyl_{(C≤12)}, or substituted aralkyl_{(C≤12)}; and
   R₂ is an organic moiety;
provided the compound is not:

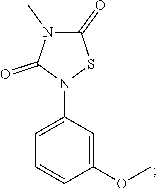 [SMILES: COc1cccc(-n2sc(=O)n(C)c2=O)c1]

or a pharmaceutically acceptable salt thereof.

In some embodiments, R₁ is alkyl_{(C≤12)} or substituted alkyl_{(C≤12)} such as methyl. In some embodiments, R₂ is an organic moiety selected from a linking group selected from —C(O)—, —C(S)—, —C(O)O—, —C(S)O—, —OC(O)—, —OC(S)—, —C(O)NR_{b}—, —C(S)NR_{b}—, —NR_{b}C(O)—, —NR_{b}C(S)—, —OC(O)O—, —OC(S)O—, —NR_{b}C(O)NR_{b}—, —NR_{b}C(S)NR_{b}—, —S(O)ₓ—, —OS(O)ₓO—, —OS(O)ₓ—, —S(O)ₓO—, and a combination thereof; wherein: x is 0, 1, or 2; and R_{b} is hydrogen, alkyl_{(C≤6)}, or substituted alkyl_{(C≤6)}, a small molecule targeting agent, hydrogen, amino, cyano, halo, hydroxy, nitro, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, alkylamino_{(C≤12)}, dialkylamino_{(C≤12)}, cycloalkylamino_{(C≤12)}, cycloalkyl(alkyl)amino_{(C≤12)}, dicycloalkylamino_{(C≤12)}, alkoxy_{(C≤12)}, aryloxy_{(C≤12)}, acyloxy_{(C≤12)}, acyl_{(C≤12)}, amido_{(C≤12)}, alkylsulfonyl_{(C≤12)}, arylsulfonyl_{(C≤12)}, or a substituted version thereof, or a combination thereof. In some embodiments, R₂ is alkyl_{(C≤12)}, substituted alkyl_{(C≤12)}, amido_{(C≤12)}, substituted amido_{(C≤12)}, or a combination thereof. In some embodiments, R₂ is substituted alkyl_{(C≤12)}. In some embodiments, R₂ is a combination of alkyl_{(C≤12)} and amido_{(C≤12)} such as —CH₂CH₂CH₂NHC(O)Ph. In some embodiments, the compounds are further defined as:

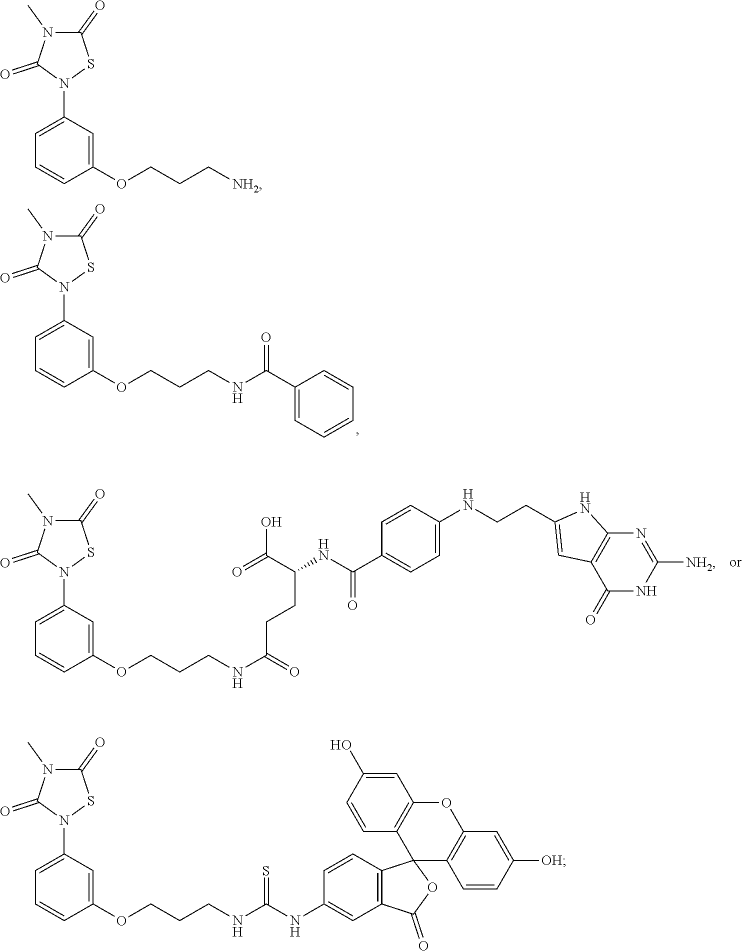

or a pharmaceutically acceptable salt thereof.

In still yet another aspect, the present disclosure provides pharmaceutical compositions comprising a compound described herein and a pharmaceutically acceptable excipient.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula does not mean that it cannot also belong to another generic formula.

### BRIEF DESCRIPTION OF THE DRAWINGS

The following drawings form part of the present specification and are included to further demonstrate certain aspects of the present disclosure. The disclosure may be better understood by reference to one of these drawings in combination with the detailed description of specific embodiments presented herein.

FIGS. 1A-B. Novel Rb1 modulator AP-3-84 induces cell death specifically in macrophages. (FIG. 1A) Tumor PDAC cells and thioglycolate-induced macrophages were cultured with different concentrations of AP-3-84 Rb1 modulator for 16 hours with the following assessment of viability by Annexin V/DAPI staining. AP-3-84 didn't affect PDA tumor cell viability, but induced macrophage cell death in dose-dependent manner. (FIG. 1B) Thioglycollate-induced macrophages were cultured with 10 uM AP-3-84 for 4 hours and gene expression changes were studied by RNA-Seq. AP-3-84 induced strong unfolded protein and oxidative stress response with the induction of apoptosis pathway and p53 signaling.

FIGS. 2A-D. AP-3-84 significantly inhibits pancreatic tumor growth in mice in an immune-mediated manner. (FIGS. 2A-B) B6 wild-type mice were orthotopically injected with PDA T high cell line into the pancreas. T high PDA cell line was previously generated by Dr. Stanger's lab (Upenn), this tumor cell line can induce some T cell infiltration opposite to T low PDA cell line (shown in FIGS. 4A-C). Mice were treated with 7 daily AP-3-84 or vehicle starting from day 4. On day 22 pancreatic tumors were collected and compared across two groups. AP-3-84 treatment significantly reduced the tumor growth in mice. (FIG. 2C) B6 wild-type mice were injected subcutaneously with PDA T high tumor cells and treated with AP-3-84 or vehicle as in FIGS. 2A-B. Tumor growth was measured until day 31. Significant reduction of tumor growth was observed in AP-3-84-treated animals. (FIG. 2D) Same experiment as in FIGS. 2A-B was conducted in NSG immunodeficient mice. No effect of AP-3-84 treatment was observed, pointing out to the immune-mediated mechanism of AP-3-84 action

FIGS. 3A-B. AP-3-84 depletes tumor macrophages and re-shapes immune cell landscape. (FIGS. 3A-B) B6 wild-type mice were orthotopically injected with PDA T high cell line into the pancreas and treated with AP-3-84 or vehicle as in FIGS. 2A-D. Tumor cells on day 12 (FIG. 3A) or peritoneum cells on day 21 (FIG. 3B) were analyzed by flow cytometry. The inventors found that AP-3-84 effectively depletes tumor-associated macrophages in tumors and overall re-shapes the immune cell composition in the peritoneum (site of AP-3-84 injection) from macrophage towards T cell-infiltrated environment. Significant effects on activation and differentiation of immune cells in tumor and periphery were also found (not shown).

FIGS. 4A-C. AP-3-84 renders more resistant PDA tumors with low T cell infiltration into immunotherapy responsive tumors. (FIGS. 4A-C) B6 wild-type mice were orthotopically (FIG. 4A) or subcutaneously (FIGS. 4B-C) injected with PDA T low cell line (this line has very low T cell infiltration and demonstrates poor response to Immunotherapy alone). Mice were treated with AP-3-84 or vehicle as in FIGS. 2A-D. AP-3-84 treatment alone didn't dramatically affect the tumor growth in both orthotopic (FIG. 4A) or subcutaneous models (FIG. 4B). However, combining AP-3-84 with Immunotherapy treatment (anti-PD1/anti-CTLA4/anti-CD40) effectively reduced the tumor growth compared to monotherapies) and increased the responsiveness of the mice to Immunotherapy treatment (FIG. 4C).

FIG. 5. Rb1 protein map showing the major portions of the Rb1 protein.

FIG. 6. LxCxE binding pocket of the Retinoblastoma (Rb) protein

FIG. 7. E7 Complex with Rb protein.

FIG. 8. A Magnified E7 Complex with Rb protein.

FIG. 9. A Complex of the E7 and its interaction with Rb protein.

FIG. 10. Inhibition of E7 mediated e2F release from GST-Rb

FIG. 11. E7 binding to Rb 379-928

FIG. 12. Rb C706A Mutation Study

FIG. 13. Binding of Rb mutants to E7, FL-E2F1, MDM2, and HDAC1.

FIG. 14. Protein Fragments and their involvement in binding

FIG. 15. Protein binding to different Rb fragments

FIG. 16. AP-3-84 inhibition of HDAC1 binding to Rb fragments

FIG. 17. Binding of HDAC1, e2F1, and MDM2 to Rb

FIG. 18. Mass analysis of AP-3-84 treated proteins after fragmentation

FIG. 19. Mechanism of AP-3-84 binding proposed and iodoacetamide blocking study

FIGS. 20A & 20B. Spatial distribution of RB1 in THP-1 cells. A. Confocal image of THP-1 cells indicates the presence of RB1 protein within the cytosol. THP-1 cells stained for DNA (blue), actin (rhodamine phalloidin, red) and RB1 protein (green) (upper panel magnification—63×; lower panel showed the magnified field of the yellow box area. B. Western blot of RB1 protein in cytosolic/Nuclear versus mitochondrial fraction indicates the presence of RB1 protein in mitochondria.

FIGS. 21A & 21B. AP-3-84 treatment induced cell death in different AML cell lines. A. Graphs indicates the total percentage of cell death (annexin V+ and AnnexinV+Live-Dead+ cells) in THP-1, AML-193 and Kasumi-1 cell line after treatment of different concentration od AP-3-84. B. Cell survival were analyzed after AP-3-84 treatment by Amalar blue assay.

FIG. 22A-22C. AP-3-84 treatment does not induce cell death or alter cell cycle in cancer cells. A. Graphs indicates the total percentage of cell death (annexin V+ and AnnexinV+Live-Dead+ cells) in PMA treated THP-1 cells. AP-3-84 has minor effect on PMA treated THP-1 cells. B. AP-3-84 does not induce cell death in ID8 cells. C. Histogram indicates cell proliferation, measured by CSFE dye dilution in presence of AP-3-84 and other inhibitors. Bar diagram indicates average MFI of CFSE dye in presence of different inhibitors.

FIG. 23A-23C. AP-3-84 treatment induced ER stress response-dependent apoptosis in THP-1 cell line. A. Western blot data indicates induction of Cleaved-PARP, Noxa and Cleaved-caspase 3 upon treatment with AP-3-84 (10 uM). B. Induction of ATF4, ATF3 and peIF2a after AP-3-84 treatment indicates the ER-stress and unfolded protein response as an early event in apoptosis induction. C. Bax Bcl-2 and Bcl-xl does not get altered after AP-3-84 treatment.

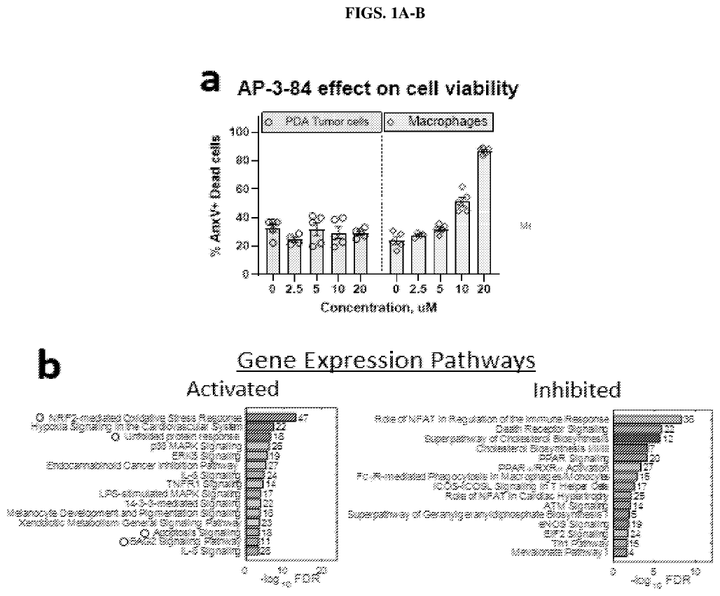

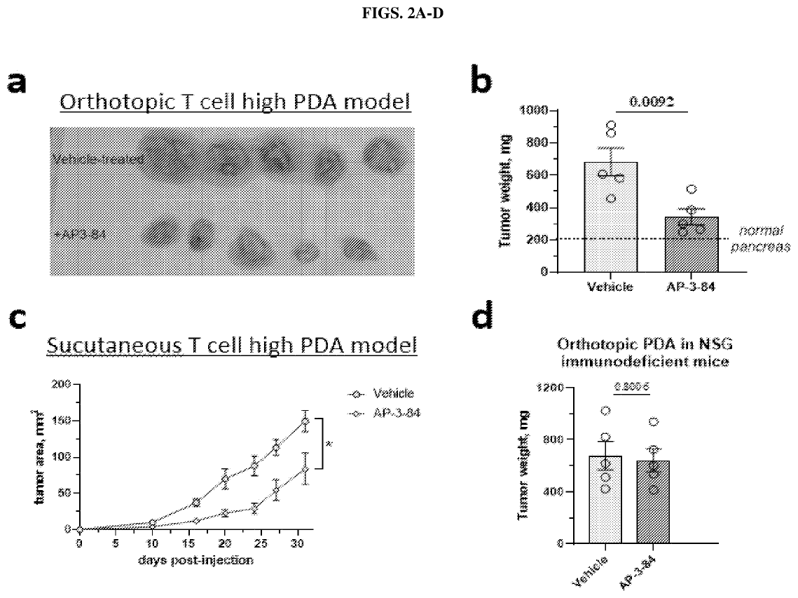

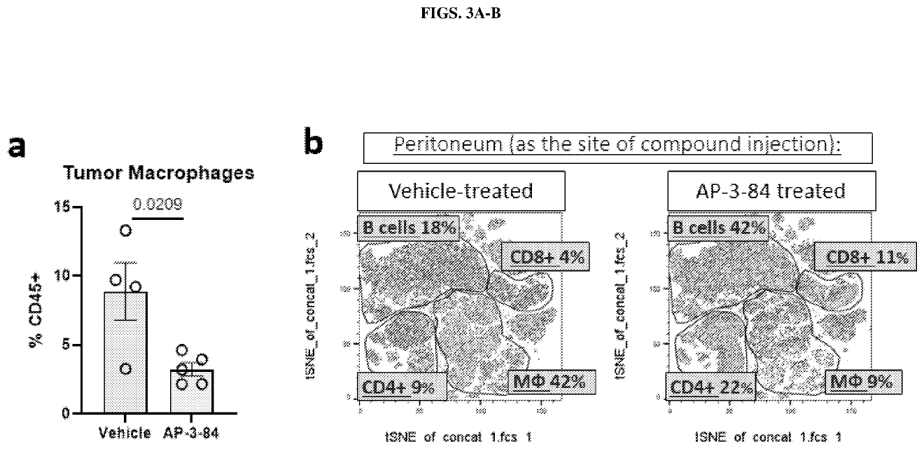

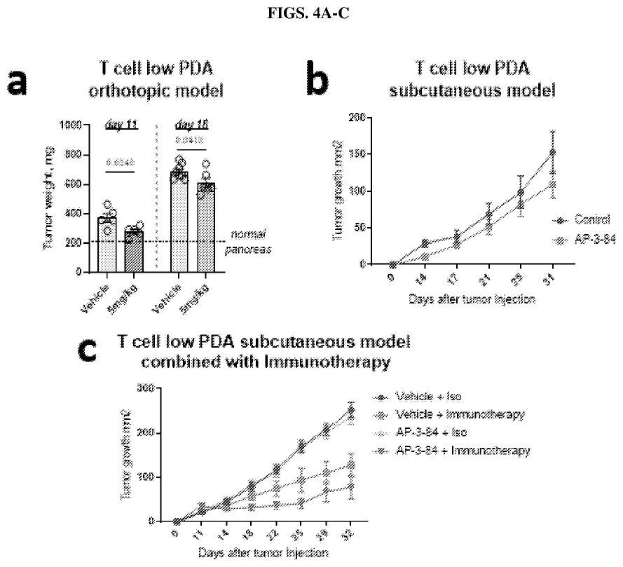

### DESCRIPTION OF ILLUSTRATIVE EMBODIMENTS

In some aspects of the present disclosure, the disclosure provides compounds which may be useful in the treatment of cancer. In particular, the compounds are envisioned to have utility against pancreatic cancer. Retinoblastoma (Rb1) protein is a known tumor suppressor protein. In addition, there is accumulating evidence for its role in immune cell response as well, including regulation of apoptosis in myeloid cells. However, the mechanisms of this phenomenon remain not clear. Here, the inventors show that compounds targeting Rb1 induce apoptosis of tumor-associated macrophages (TAMs) in a model of PDA, induces T cell infiltration of the tumor and decreases tumor burden. Such compounds are proposed for treatment of tumors by direct injection of the compound into the tumor or when administered systemically. Furthermore, the inventors have also tested and show the compounds to be effective in the treatment of leukemia such as AML. These and other aspects of the disclosure are described in detail below.

### I. DEFINITIONS

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl. —Br or —I; “amino” means —NH₂; “hydroxyamino” means —NHOH; “nitro” means —NO₂; imino means ═NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means —OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “—” means a single bond, “═” means a double bond, and “≡” means triple bond. The symbol “ [symbol] ” represents an optional bond, which if present is either single or double. The symbol “ [symbol] ” represents a single bond or a double bond. Thus, the formula

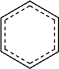

covers, for example,

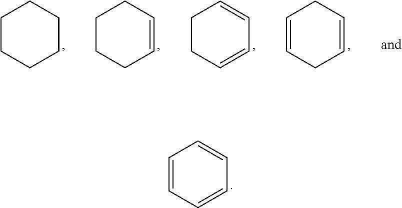

And it is understood that no one such ring atom forms part of more than one double bond. Furthermore, it is noted that the covalent bond symbol “—”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.,

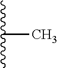

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

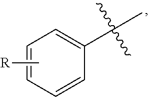

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

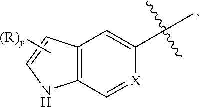

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl(ci-4)”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino(c₆) group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic 71 system. An aromatic compound or chemical group may be depicted as a single resonance structure; however, depiction of one resonance structure is taken to also refer to any other resonance structure. For example:

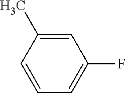

is also taken to refer to

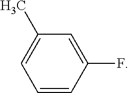

Aromatic compounds may also be depicted using a circle to represent the delocalized nature of the electrons in the fully conjugated cyclic 7L system, two non-limiting examples of which are shown below;

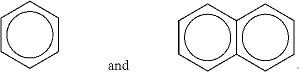

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂(i-Pr, ⁱPr or isopropyl), —CH₂CH₂CH₂CH₃ (n-Bu), —CH(CH₃)CH₂CH₃ (sec-butyl), —CH₂CH(CH₃)₂(isobutyl), —C(CH₃)₃(tert-butyl, i-butyl, i-Bu or ^{t}Bu), and —CH₂C(CH₃)₃(neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and =C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. If more than one ring is present, the rings may be fused, bridged, or spirocyclic. Non-limiting examples include: —CH(CH₂)₂(cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

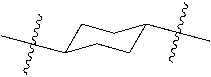 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂—are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

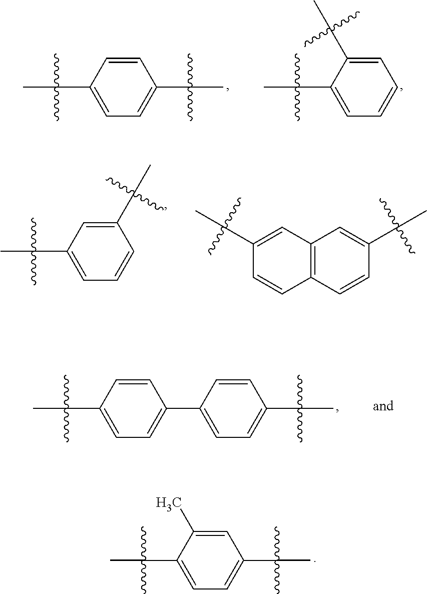

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings may be fused, bridged, or spirocyclic. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, tetrahydropyridinyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂(isopropoxy), or —OC(CH₃)₃(tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients.

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” means a compound that is convertible in vivo metabolically into an active pharmaceutical ingredient of the present invention. The prodrug itself may or may not have activity in its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### II. COMPOUNDS OF THE PRESENT DISCLOSURE

In the present disclosure, anticancer agents are described. The compounds in this disclosure can be prepared according to standard methods in the Appendix. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein. In some embodiments, the present disclosure contains a compound of the formula as described in Table 1.

TABLE 1
Compounds of the Present Disclosure

| Compound ID | Structure |
|---|---|
| Tideglusib | 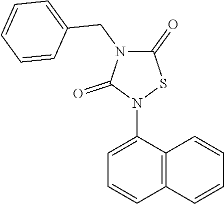 [SMILES: O=c1sn(-c2cccc3ccccc23)c(=O)n1Cc1ccccc1] |
| AP-08-231 | 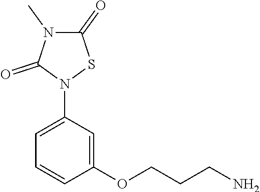 [SMILES: Cn1c(=O)sn(-c2cccc(OCCCN)c2)c1=O] |
| AP-08-237 | 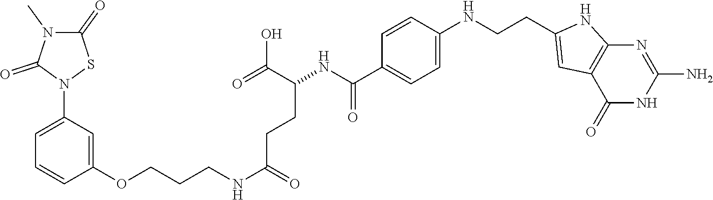 [SMILES: Cn1c(=O)sn(-c2cccc(OCCCNC(=O)CC[C@@H](NC(=O)c3ccc(NCCc4cc5c(=O)[nH]c(N)nc5[nH]4)cc3)C(=O)O)c2)c1=O] |
| AP-08-239 | 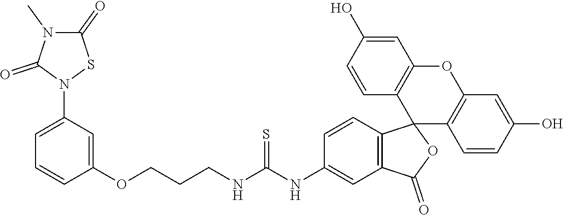 [SMILES: Cn1c(=O)sn(-c2cccc(OCCCNC(=S)Nc3ccc4c(c3)C(=O)OC43c4ccc(O)cc4Oc4cc(O)ccc43)c2)c1=O] |
| AP-03-084 | 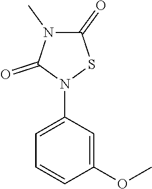 [SMILES: COc1cccc(-n2sc(=O)n(C)c2=O)c1] |
| AP-08-238 | 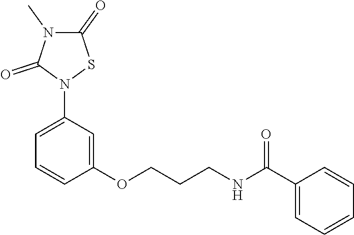 [SMILES: Cn1c(=O)sn(-c2cccc(OCCCNC(=O)c3ccccc3)c2)c1=O] |
| SA-01-048 | 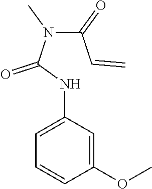 [SMILES: C=CC(=O)N(C)C(=O)Nc1cccc(OC)c1] |

The compounds described in this disclosure may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. The compounds may occur as racemates and racemic mixtures, single enantiomers, diastereomeric mixtures and individual diastereomers. In some embodiments, a single diastereomer is obtained. The chiral centers of the present disclosure can have the S or the R configuration.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C. Similarly, it is contemplated that one or more carbon atom(s) of a compound of the present disclosure may be replaced by a silicon atom(s). Furthermore, it is contemplated that one or more oxygen atom(s) of the compounds may be replaced by a sulfur or selenium atom(s).

The compounds may also have the advantage that they may be more efficacious than, be less toxic than, be longer acting than, be more potent than, produce fewer side effects than, be more easily absorbed than, and/or have a better pharmacokinetic profile (e.g., higher oral bioavailability and/or lower clearance) than, and/or have other useful pharmacological, physical, or chemical advantages over, compounds known in the prior art for use in the indications stated herein.

Compounds of the present disclosure may also exist in prodrug form. Since prodrugs are known to enhance numerous desirable qualities of pharmaceuticals (e.g., solubility, bioavailability, manufacturing, etc.), the compounds employed in some methods of the disclosure may, if desired, be delivered in prodrug form. Thus, the disclosure contemplates prodrugs of compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a subject, cleaves to form a hydroxy, amino, or carboxylic acid, respectively.

It should be recognized that the particular anion or cation forming a part of any salt of this disclosure is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

### III. HYPERPROLIFERATIVE DISEASES

### A. Cancers

While hyperproliferative diseases can be associated with any disease which causes a cell to begin to reproduce uncontrollably, cancer is the common example. One of the key elements of cancer is that the cell's normal apoptotic cycle is interrupted and thus agents that lead to apoptosis of the cell are important therapeutic agents for treating these diseases. In this disclosure, the compounds of the present disclosure have been shown to lead to cellular apoptosis and as such can potentially be used to treat a variety of types of cancer lines. As such, the compounds of the present disclosure may be used to effectively treat cancers such as pancreatic cancer. In various aspects, it is anticipated that compounds of the present disclosure may be used to treat virtually any malignancy.

Cancer cells that may be treated with the compounds of the present disclosure according to the embodiments include but are not limited to cells from the bladder, blood, bone, bone marrow, brain, breast, colon, esophagus, gastrointestine, gum, head, kidney, liver, lung, nasopharynx, neck, oral, ovary, prostate, skin, stomach, pancreas, testis, tongue, cervix, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; malignant histiocytosis; multiple myeloma; mast cell sarcoma; immunoproliferative small intestinal disease; leukemia; lymphoid leukemia; plasma cell leukemia; erythroleukeinia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia. In certain aspects, the tumor may comprise an osteosarcoma, angiosarcoma, rhabdosarcoma, leiomyosarcoma, Ewing sarcoma, glioblastoma, neuroblastoma, or leukemia.

### B. Pancreatic Cancer

A cancer of particular interest with respect to the present disclosure is pancreatic cancer. Signs and symptoms of the most-common form of pancreatic cancer—pancreatic adenocarcinoma—may include yellow skin, abdominal or back pain, unexplained weight loss, light-colored stools, dark urine, and loss of appetite. Usually, no symptoms are seen in the disease's early stages, and symptoms that are specific enough to suggest pancreatic cancer typically do not develop until the disease has reached an advanced stage. By the time of diagnosis, pancreatic cancer has often spread to other parts of the body.

Pancreatic cancer rarely occurs before the age of 40, and more than half of cases of pancreatic adenocarcinoma occur in those over 70. Risk factors for pancreatic cancer include tobacco smoking, obesity, diabetes, and certain rare genetic conditions. About 25% of cases are linked to smoking, and 5-10% are linked to inherited genes. Pancreatic cancer is usually diagnosed by a combination of medical imaging techniques such as ultrasound or computed tomography, blood tests, and examination of tissue samples (biopsy). The disease is divided into stages, from early (stage I) to late (stage IV). Screening the general population has not been found to be effective.

The risk of developing pancreatic cancer is lower among non-smokers, and people who maintain a healthy weight and limit their consumption of red or processed meat. Smokers' chances of developing the disease decrease if they stop smoking and almost return to that of the rest of the population after 20 years. Pancreatic cancer can be treated with surgery, radiotherapy, chemotherapy, palliative care, or a combination of these. Treatment options are partly based on the cancer stage. Surgery is the only treatment that can cure pancreatic adenocarcinoma, and may also be done to improve quality of life without the potential for cure. Pain management and medications to improve digestion are sometimes needed. Early palliative care is recommended even for those receiving treatment that aims for a cure.

The many types of pancreatic cancer can be divided into two general groups. The vast majority of cases (about 95%) occur in the part of the pancreas that produces digestive enzymes, known as the exocrine component. Several subtypes of exocrine pancreatic cancers are described, but their diagnosis and treatment have much in common. The small minority of cancers that arise in the hormone-producing (endocrine) tissue of the pancreas have different clinical characteristics and are called pancreatic neuroendocrine tumors, sometimes abbreviated as “PanNETs”. Both groups occur mainly (but not exclusively) in people over 40, and are slightly more common in men, but some rare subtypes mainly occur in women or children.

Exocrine cancers. The exocrine group is dominated by pancreatic adenocarcinoma (variations of this name may add “invasive” and “ductal”), which is by far the most common type, representing about 85% of all pancreatic cancers. Nearly all these start in the ducts of the pancreas, as pancreatic ductal adenocarcinoma (PDAC). This is despite the fact that the tissue from which it arises—the pancreatic ductal epithelium—represents less than 10% of the pancreas by cell volume, because it constitutes only the ducts (an extensive but capillary-like duct-system fanning out) within the pancreas. This cancer originates in the ducts that carry secretions (such as enzymes and bicarbonate) away from the pancreas. About 60-70% of adenocarcinomas occur in the head of the pancreas.

The next-most common type, acinar cell carcinoma of the pancreas, arises in the clusters of cells that produce these enzymes, and represents 5% of exocrine pancreas cancers. Like the ‘functioning’ endocrine cancers described below, acinar cell carcinomas may cause over-production of certain molecules, in this case digestive enzymes, which may cause symptoms such as skin rashes and joint pain.

Cystadenocarcinomas account for 1% of pancreatic cancers, and they have a better prognosis than the other exocrine types.

Pancreatoblastoma is a rare form, mostly occurring in childhood, and with a relatively good prognosis. Other exocrine cancers include adenosquamous carcinomas, signet ring cell carcinomas, hepatoid carcinomas, colloid carcinomas, undifferentiated carcinomas, and undifferentiated carcinomas with osteoclast-like giant cells. Solid pseudopapillary tumor is a rare low-grade neoplasm that mainly affects younger women, and generally has a very good prognosis.

Pancreatic mucinous cystic neoplasms are a broad group of pancreas tumors that have varying malignant potential. They are being detected at a greatly increased rate as CT scans become more powerful and common, and discussion continues as how best to assess and treat them, given that many are benign.

Neuroendocrine. The small minority of tumors that arise elsewhere in the pancreas are mainly pancreatic neuroendocrine tumors (PanNETs). Neuroendocrine tumors (NETs) are a diverse group of benign or malignant tumors that arise from the body's neuroendocrine cells, which are responsible for integrating the nervous and endocrine systems. NETs can start in most organs of the body, including the pancreas, where the various malignant types are all considered to be rare. PanNETs are grouped into ‘functioning’ and ‘nonfunctioning’ types, depending on the degree to which they produce hormones. The functioning types secrete hormones such as insulin, gastrin, and glucagon into the bloodstream, often in large quantities, giving rise to serious symptoms such as low blood sugar, but also favoring relatively early detection. The most common functioning PanNETs are insulinomas and gastrinomas, named after the hormones they secrete. The nonfunctioning types do not secrete hormones in a sufficient quantity to give rise to overt clinical symptoms, so nonfunctioning PanNETs are often diagnosed only after the cancer has spread to other parts of the body.

As with other neuroendocrine tumors, the history of the terminology and classification of PanNETs is complex. PanNETs are sometimes called “islet cell cancers”, though they are now known to not actually arise from islet cells as previously thought.

Risk factors for pancreatic adenocarcinoma include:
Age, sex, and ethnicity—the risk of developing pancreatic cancer increases with age. Most cases occur after age 65, while cases before age 40 are uncommon. The disease is slightly more common in men than in women. In the United States, it is over 1.5 times more common in African Americans, though incidence in Africa is low.
Smoking—Cigarette smoking is the best-established avoidable risk factor for pancreatic cancer, approximately doubling risk among long-term smokers, the risk increasing with the number of cigarettes smoked and the years of smoking. The risk declines slowly after smoking cessation, taking some 20 years to return to almost that of nonsmokers.
Obesity—a body mass index greater than 35 increases relative risk by about half.
Family history—5-10% of pancreatic cancer cases have an inherited component, where people have a family history of pancreatic cancer. The risk escalates greatly if more than one first-degree relative had the disease, and more modestly if they developed it before the age of 50. Most of the genes involved have not been identified. Hereditary pancreatitis gives a greatly increased lifetime risk of pancreatic cancer of 30-40% to the age of 70. Screening for early pancreatic cancer may be offered to individuals with hereditary pancreatitis on a research basis. Some people may choose to have their pancreas surgically removed to prevent cancer from developing in the future.
Hereditary risk—Pancreatic cancer has been associated with these other rare hereditary syndromes: Peutz-Jeghers syndrome due to mutations in the STK11 tumor suppressor gene (very rare, but a very strong risk factor); dysplastic nevus syndrome (or familial atypical multiple mole and melanoma syndrome, FAMMM-PC) due to mutations in the CDKN2A tumor suppressor gene; autosomal recessive ataxia-telangiectasia and autosomal dominantly inherited mutations in the BRCA2 and PALB2 genes; hereditary non-polyposis colon cancer (Lynch syndrome); and familial adenomatous polyposis. PanNETs have been associated with multiple endocrine neoplasia type 1 (MEN1) and von Hippel Lindau syndromes.
Chronic pancreatitis—Pancreatitis appears to almost triple risk, and as with diabetes, new-onset pancreatitis may be a symptom of a tumor. The risk of pancreatic cancer in individuals with familial pancreatitis is particularly high.
Diabetes—Diabetes mellitus is a risk factor for pancreatic cancer and (as noted in the Signs and symptoms section) new-onset diabetes may also be an early sign of the disease. People who have been diagnosed with type 2 diabetes for longer than 10 years may have a 50% increased risk, as compared with individuals without diabetes. In 2021, Venturi reported that pancreas is able to absorb in great quantity radioactive cesium (Cs-134 and Cs-137) causing chronic pancreatitis and probably pancreatic cancer with damage of pancreatic islands, causing Type 3c (pancreatogenic) diabetes. Chronic pancreatitis, pancreatic cancer and diabetes mellitus increased in contaminated population, particularly children and adolescents, after Fukushima and Chernobyl nuclear incidents. At the same time, worldwide pancreatic diseases, diabetes and environmental radiocesium are increasing.
Food—Specific types of food (as distinct from obesity) have not been clearly shown to increase the risk of pancreatic cancer. Dietary factors for which some evidence shows slightly increased risk include processed meat, red meat, and meat cooked at very high temperatures (e.g., by frying, broiling, or grilling).

The symptoms of pancreatic adenocarcinoma do not usually appear in the disease's early stages, and they are not individually distinctive to the disease. The symptoms at diagnosis vary according to the location of the cancer in the pancreas, which anatomists divide (from left to right on most diagrams) into the thick head, the neck, and the tapering body, ending in the tail.

Regardless of a tumor's location, the most common symptom is unexplained weight loss, which may be considerable. A large minority (between 35% and 47%) of people diagnosed with the disease will have had nausea, vomiting, or a feeling of weakness. Tumors in the head of the pancreas typically also cause jaundice, pain, loss of appetite, dark urine, and light-colored stools. Tumors in the body and tail typically also cause pain.

People sometimes have recent onset of atypical type 2 diabetes that is difficult to control, a history of recent but unexplained blood vessel inflammation caused by blood clots (thrombophlebitis) known as Trousseau sign, or a previous attack of pancreatitis. A doctor may suspect pancreatic cancer when the onset of diabetes in someone over 50 years old is accompanied by typical symptoms such as unexplained weight loss, persistent abdominal or back pain, indigestion, vomiting, or fatty feces. Jaundice accompanied by a painlessly swollen gallbladder (known as Courvoisier's sign) may also raise suspicion, and can help differentiate pancreatic cancer from gallstones.

Medical imaging techniques, such as computed tomography (CT scan) and endoscopic ultrasound (EUS) are used both to confirm the diagnosis and to help decide whether the tumor can be surgically removed (its “resectability”). On contrast CT scan, pancreatic cancer typically shows a gradually increasing radiocontrast uptake, rather than a fast washout as seen in a normal pancreas or a delayed washout as seen in chronic pancreatitis. Magnetic resonance imaging and positron emission tomography may also be used, and magnetic resonance cholangiopancreatography may be useful in some cases. Abdominal ultrasound is less sensitive and will miss small tumors, but can identify cancers that have spread to the liver and build-up of fluid in the peritoneal cavity (ascites). It may be used for a quick and cheap first examination before other techniques.

A biopsy by fine needle aspiration, often guided by endoscopic ultrasound, may be used where there is uncertainty over the diagnosis, but a histologic diagnosis is not usually required for removal of the tumor by surgery to go ahead.

Liver function tests can show a combination of results indicative of bile duct obstruction (raised conjugated bilirubin, γ-glutamyl transpeptidase and alkaline phosphatase levels). CA19-9 (carbohydrate antigen 19.9) is a tumor marker that is frequently elevated in pancreatic cancer. However, it lacks sensitivity and specificity, not least because 5% of people lack the Lewis (a) antigen and cannot produce CA19-9. It has a sensitivity of 80% and specificity of 73% in detecting pancreatic adenocarcinoma and is used for following known cases rather than diagnosis.

### C. Leukemia

Clinically and pathologically, leukemia is subdivided into a variety of large groups. The first division is between its acute and chronic forms. Acute leukemia is characterized by a rapid increase in the number of immature blood cells. Crowding due to such cells makes the bone marrow unable to produce healthy blood cells. Immediate treatment is required in acute leukemia due to the rapid progression and accumulation of the malignant cells, which then spill over into the bloodstream and spread to other organs of the body. Acute forms of leukemia are the most common forms of leukemia in children. Chronic leukemia is characterized by the excessive build up of relatively mature, but still abnormal, white blood cells. Typically taking months or years to progress, the cells are produced at a much higher rate than normal, resulting in many abnormal white blood cells. Whereas acute leukemia must be treated immediately, chronic forms are sometimes monitored for some time before treatment to ensure maximum effectiveness of therapy. Chronic leukemia mostly occurs in older people, but can theoretically occur in any age group.

Additionally, the diseases are subdivided according to which kind of blood cell is affected. This split divides leukemias into lymphoblastic or lymphocytic leukemias and myeloid or myelogenous leukemias. In lymphoblastic or lymphocytic leukemias, the cancerous change takes place in a type of marrow cell that normally goes on to form lymphocytes, which are infection-fighting immune system cells. Most lymphocytic leukemias involve a specific subtype of lymphocyte, the B cell. In myeloid or myelogenous leukemias, the cancerous change takes place in a type of marrow cell that normally goes on to form red blood cells, some other types of white cells, and platelets.

Combining these two classifications provides a total of four main categories. Within each of these four main categories, there are typically several subcategories. Finally, some rarer types are usually considered to be outside of this classification scheme:
Acute lymphoblastic leukemia (ALL) is the most common type of leukemia in young children. This disease also affects adults, especially those age 65 and older. Standard treatments involve chemotherapy and radiotherapy. The survival rates vary by age: 85% in children and 50% in adults. Subtypes include precursor B acute lymphoblastic leukemia, precursor T acute lymphoblastic leukemia, Burkit's leukemia, and acute biphenotypic leukemia.
Chronic lymphocytic leukemia (CLL) most often affects adults over the age of 55. It sometimes occurs in younger adults, but it almost never affects children. Two-thirds of affected people are men. The five-year survival rate is 75%. It is incurable, but there are many effective treatments. One subtype is B-cell prolymphocytic leukemia, a more aggressive disease.
Acute myelogenous leukemia or acute myeloid leukemia (AML) occurs more commonly in adults than in children, and more commonly in men than women. AML is treated with chemotherapy. The five-year survival rate is 40%, except for acute promyelocytic leukemia (APL), which is over 90%. Subtypes of AML include acute promyelocytic leukemia (APL), acute myeloblastic leukemia, and acute megakaryoblastic leukemia.
Chronic myelogenous leukemia (CML) occurs mainly in adults; a very small number of children also develop this disease. Treatment is with imatinib (Gleevec in United States, Glivec in Europe) or other drugs. The five-year survival rate is 90%. One subtype is chronic monocytic leukemia.
Hairy cell leukemia (HCL) is sometimes considered a subset of chronic lymphocytic leukemia, but does not fit neatly into this pattern. About 80% of affected people are adult men. No cases in children have been reported. HCL is incurable, but easily treatable. Survival is 96% to 100% at ten years.
T-cell prolymphocytic leukemia (T-PLL) is a very rare and aggressive leukemia affecting adults; somewhat more men than women are diagnosed with this disease. Despite its overall rarity, it is also the most common type of mature T cell leukemia; nearly all other leukemias involve B cells. It is difficult to treat, and the median survival is measured in months.
Large granular lymphocytic leukemia may involve either T-cells or NK cells; like hairy cell leukemia, which involves solely B cells, it is a rare and indolent (not aggressive) leukemia.

Adult T-cell leukemia is caused by human T-lymphotropic virus (HTLV), a virus similar to HIV. Like HIV, HTLV infects CD4+ T-cells and replicates within them; however, unlike HIV, it does not destroy them. Instead, HTLV “immortalizes” the infected T-cells, giving them the ability to proliferate abnormally. Human T cell lymphotropic virus types I and II (HTLV-1/II) are endemic in certain areas of the world.

### IV. PHARMACEUTICAL FORMULATIONS AND ROUTES OF ADMINISTRATION

For administration to a mammal in need of such treatment, the compounds of the present disclosure in a therapeutically effective amount are ordinarily combined with one or more excipients appropriate to the indicated route of administration. The compounds of the present disclosure may be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol, and tableted or encapsulated for convenient administration. Alternatively, the compounds of the present disclosure may be dissolved in water, polyethylene glycol, propylene glycol, ethanol, corn oil, cottonseed oil, peanut oil, sesame oil, benzyl alcohol, sodium chloride, and/or various buffers. Other excipients and modes of administration are well and widely known in the pharmaceutical art.

The pharmaceutical compositions useful in the present disclosure may be subjected to conventional pharmaceutical operations such as sterilization and/or may contain conventional pharmaceutical carriers and excipients such as preservatives, stabilizers, wetting agents, emulsifiers, buffers, etc.

The compounds of the present disclosure may be administered by a variety of methods, e.g., orally or by injection (e.g., subcutaneous, intratumoral, intravenous, intraperitoneal, etc.). Depending on the route of administration, the compounds of the present disclosure may be coated in a material to protect the compound from the action of acids and other natural conditions which may inactivate the compound. They may also be administered by continuous perfusion/infusion of a disease or wound site.

To administer the therapeutic compound by other than parenteral administration, it may be necessary to coat the compounds of the present disclosure with, or co-administer the compounds of the present disclosure with, a material to prevent its inactivation. For example, the therapeutic compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds of the present disclosure derivatives may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion are also envisioned. In all cases, the composition must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

Sterile injectable solutions can be prepared by incorporating the compounds of the present disclosure in the required amount in an appropriate solvent with one or a combination of ingredients enumerated above, as required, followed by filtered sterilization. Generally, dispersions are prepared by incorporating the therapeutic compound into a sterile carrier which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient (i.e., the therapeutic compound) plus any additional desired ingredient from a previously sterile-filtered solution thereof.

The compounds of the present disclosure can be orally administered, for example, with an inert diluent or an assimilable edible carrier. The therapeutic compound and other ingredients may also be enclosed in a hard- or soft-shell gelatin capsule, compressed into tablets, or incorporated directly into the subject's diet. For oral therapeutic administration, the compounds of the present disclosure may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the compounds of the present disclosure in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of the compounds of the present disclosure calculated to produce the desired therapeutic effect in association with the required pharmaceutical carrier. The specification for the dosage unit forms of the disclosure are dictated by and directly dependent on (a) the unique characteristics of the compounds of the present disclosure described in this disclosure and the particular therapeutic effect to be achieved, and (b) the limitations inherent in the art of compounding such a therapeutic compound for the treatment of a selected condition in a patient.

The therapeutic compound may also be administered topically to the skin, eye, or mucosa. Alternatively, if local delivery to the lungs is desired the therapeutic compound may be administered by inhalation in a dry-powder or aerosol formulation.

The compounds of the present disclosure describe in this disclosure are administered at a therapeutically effective dosage sufficient to treat a condition associated with a condition in a patient. For example, the efficacy of the compounds of the present disclosure can be evaluated in an animal model system that may be predictive of efficacy in treating the disease in humans, such as the model systems shown in the examples and drawings.

In some embodiments, the actual dosage amount of the compounds of the present disclosure of the present disclosure or composition comprising the compounds of the present disclosure of the present disclosure administered to a subject is determined by physical and physiological factors such as age, sex, body weight, severity of condition, the type of disease being treated, previous or concurrent therapeutic interventions, idiopathy of the subject and on the route of administration. These factors may be used by a skilled artisan to determine the appropriate dosage amount. The practitioner responsible for administration will typically determine the concentration of active ingredient(s) in a composition and appropriate dose(s) for the individual subject. The dosage may be adjusted by the individual physician in the event of any complication.

An effective amount typically will vary from about 2 mg/kg to about 50 mg/kg, in one or more dose administrations daily, for one or several days (depending of course of the mode of administration and the factors discussed above). In some particular embodiments, the amount is less than 5,000 mg per day with a range of 100 mg to 4500 mg per day.

In some embodiments, the effective amount is less than 10 mg/kg/day, less than 100 mg/kg/day, less than 250 mg/kg/day, less than 100 mg/kg/day, less than 50 mg/kg/day, less than 25 mg/kg/day or less than 10 mg/kg/day. Alternatively, in some embodiments, the range is 1 mg/kg/day to 200 mg/kg/day.

In other non-limiting examples, a dose may also comprise from about 10 mg/kg/body weight, about 100 mg/kg/body weight, about 10 g/kg/body weight, about 5 g/kg/body weight, or more per administration, and any range derivable therein. In non-limiting examples of a derivable range from the numbers listed herein, a range of about 1 mg/kg/body weight to about 100 mg/kg/body weight, about 5 g/kg/body weight to about 10 g/kg/body weight, etc., can be administered, based on the numbers described above.

In certain embodiments, a pharmaceutical composition of the present disclosure may comprise, for example, at least about 0.1% of a compound. In other embodiments, the compound of the present disclosure may comprise between about 0.25% to about 75% of the weight of the unit, or between about 25% to about 60%, or between about 1% to about 10%, for example, and any range derivable therein.

Single or multiple doses of the agents are contemplated. Desired time intervals for delivery of multiple doses can be determined by one of ordinary skill in the art employing no more than routine experimentation. As an example, subjects may be administered two doses daily at approximately 12-hour intervals. In some embodiments, the agent is administered once a day.

The compounds of the present disclosure may be administered on a routine schedule. As used herein a routine schedule refers to a predetermined designated period of time. The routine schedule may encompass periods of time which are identical or that differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the disclosure provides that the agent(s) may be taken orally and that the timing of which is or is not dependent upon food intake. Thus, for example, the agent can be taken every morning and/or every evening, regardless of when the subject has eaten or will eat. In other embodiments, the disclosure is taken as a dietary supplement. In some embodiments, the compounds of the present disclosure are taken before the onset of the tumor as a prophylaxis measure. In other embodiments, the compounds of the present disclosure are taken as a treatment option for use as an antiproliferative agent.

### V. Combination Therapy

In addition to being used as a monotherapy, the compounds of the present disclosure described in the present disclosure may also find use in combination therapies. Effective combination therapy may be achieved with a single composition or pharmacological formulation that includes both agents, or with two distinct compositions or formulations, administered at the same time, wherein one composition includes a compound of the present disclosure, and the other includes the second agent(s). The other therapeutic modality may be administered before, concurrently with, or following administration of the compounds of the present disclosure. The therapy using the compounds of the present disclosure may precede or follow administration of the other agent(s) by intervals ranging from minutes to weeks. In embodiments where the other agent and the compounds of the present disclosure are administered separately, one would generally ensure that a significant period of time did not expire between the time of each delivery, such that each agent would still be able to exert an advantageously combined effect. In such instances, it is contemplated that one would typically administer the compounds of the present disclosure and the other therapeutic agent within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of a compound of the present disclosure, or the other agent will be desired. In this regard, various combinations may be employed. By way of illustration, where the compound of the present disclosure is “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated. Non-limiting examples of pharmacological agents that may be used in the present disclosure include any pharmacological agent known to be of benefit in the treatment of a cancer or hyperproliferative disorder or disease. In some embodiments, combinations of the compounds of the present disclosure with a cancer targeting immunotherapy, radiotherapy, chemotherapy, or surgery are contemplated. Also contemplated is a combination of a compound of the present disclosure with more than one of the above-mentioned methods including more than one type of a specific therapy.

The skilled artisan is directed to “Remington's Pharmaceutical Sciences” 15^{th} Edition, chapter 33, in particular pages 624-652. Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

It also should be pointed out that any of the foregoing therapies may prove useful by themselves in treating cancer.

### 1. Chemotherapy

In some embodiments, the compounds of the present disclosure can be used in conjunction with one or more additional chemotherapies. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammalI and calicheamicin omegaI1; dynemicin, including dynemicin A; bisphosphonates, such as clodronate; an esperamicin; as well as neocarzinostatin chromophore and related chromoprotein enediyne antibiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); 44inblast; vindesine; dacarbazine; mannomustine; mitobronitol; mitolactol; pipobroman; gacytosine; arabinoside (“Ara-C”); cyclophosphamide; thiotepa; taxoids, e.g., paclitaxel and doxetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

Furthermore, it is contemplated that the compounds of the present disclosure are used in combination with a chemotherapeutic agent such as gefitinib, TAE684, tivantinib, anthracyclines, taxanes, methotrexate, mitoxantrone, estramustine, doxorubicin, etoposide, vinblastine, carboplatin, vinorelbine, 5-fluorouracil, cisplatin, topotecan, ifosfamide, cyclophosphamide, epirubicin, gemcitabine, vinorelbine, irinotecan, etoposide, vinblastine, pemetrexed, melphalan, capecitabine, oxaliplatin, BRAF inhibitors, and TGF-beta inhibitors. In some embodiments, the combination therapy is designed to target a cancer such as those listed above.

### 2. Radiotherapy

In some embodiments, the compounds of the present disclosure can be used in conjunction with radiotherapy. Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Without being bound by theory, radiotherapy can increase the amount of reactive oxygen species in tumor cells. In some embodiments, the combination therapy of the compounds of the present disclosure and radiotherapy can enhance the production of reactive oxygen species and thus the anti-tumor effects of the treatment. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and can be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

### 3. Immunotherapy

In some embodiments, the compounds of the present disclosure can be used in conjunction with one or more additional immunotherapies. In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155. An alternative aspect of immunotherapy is to combine anticancer effects with immune stimulatory effects. Immune stimulating molecules also exist including: cytokines such as IL-2, IL-4, IL-6, IL-10, IL-12, GM-CSF, γ-IFN, chemokines such as MIP-1, MCP-1, IL-8 and growth factors such as FLT3 ligand. Combining immune stimulating molecules, either as proteins or using gene delivery in combination with a tumor suppressor has been shown to enhance anti-tumor effects. Moreover, antibodies against any of these compounds can be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169), cytokine therapy, e.g., interferons α, β, and γ; IL-1, GM-CSF and TNF gene therapy, e.g., TNF, IL-1, IL-2, p53 (U.S. Pat. Nos. 5,830,880 and 5,846,945) and monoclonal antibodies, e.g., anti-ganglioside GM2, anti-HER-2, anti-p185 (U.S. Pat. No. 5,824,311). It is contemplated that one or more anti-cancer therapies may be employed with the gene silencing therapies described herein.

In active immunotherapy, an antigenic peptide, polypeptide or protein, or an autologous or allogenic tumor cell composition or “vaccine” is administered, generally with a distinct bacterial adjuvant.

In adoptive immunotherapy, the patient's circulating lymphocytes, or tumor infiltrated lymphocytes, are isolated in vitro, activated by lymphokines such as IL-2 or transduced with genes for tumor necrosis, and readministered.

In some embodiments, it is contemplated that the immunotherapy is a monoclonal antibody which targets HER2/neu such trastuzumab (Herceptin®) or a similar antibody. In other embodiments, the immunotherapy can be other cancer targeting antibodies such as alemtuzumab (Campath®), bevacizumab (Avastin®), cetuximab (Eribitux®), and panitumumab (Vectibix®) or conjugated antibodies such as ibritumomab tiuxetan (Zevalin®), tositumomab (Bexxar®), brentuximab vedotin (Adcetris®), ado-trastuzumab emtansine (Kadcyla™), or denileukin dititox (Ontak®) as well as immune cell targeting antibodies such as ipilimumab (Yervoy®), tremelimumab, anti-PD-1, anti-4-1-BB, anti-GITR, anti-TIM3, anti-LAG-3, anti-TIGIT, anti-CTLA-4, or anti-LIGHT. Additionally, in some embodiments, the compounds of the present disclosure can be administered with gefitinib, TAE684, tivantinib, or combinations of these drugs. Furthermore, in some embodiments, the compounds of the present disclosure are envisioned to be used in combination therapies with dendritic cell-based immunotherapies such as Sipuleucel-T (Provenge®) or adoptive T-cell immunotherapies.

### 4. Surgery

In some embodiments, the compounds of the present disclosure can be used in conjunction with surgery. Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

### 5. Other Agents

It is contemplated that other agents may be used with the present disclosure. These additional agents include immunomodulatory agents, agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Immunomodulatory agents include tumor necrosis factor; interferon alpha, beta, and gamma; IL-2 and other cytokines; F42K and other cytokine analogs; or MIP-1, MIP-10, MCP-1, RANTES, and other chemokines. It is further contemplated that the upregulation of cell surface receptors or their ligands such as Fas/Fas ligand, DR4 or DR5/TRAIL (Apo-2 ligand) would potentiate the apoptotic inducing abilities of the present disclosure by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present disclosure to improve the anti-hyerproliferative efficacy of the treatments. Inhibitors of cell adhesion are contemplated to improve the efficacy of the present disclosure. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

There have been many advances in the therapy of cancer following the introduction of cytotoxic chemotherapeutic drugs. However, one of the consequences of chemotherapy is the development/acquisition of drug-resistant phenotypes and the development of multiple drug resistance. The development of drug resistance remains a major obstacle in the treatment of such tumors and therefore, there is an obvious need for alternative approaches such as gene therapy.

Another form of therapy for use in conjunction with chemotherapy, radiation therapy or biological therapy includes hyperthermia, which is a procedure in which a patient's tissue is exposed to high temperatures (up to 106° F.). External or internal heating devices may be involved in the application of local, regional, or whole-body hyperthermia. Local hyperthermia involves the application of heat to a small area, such as a tumor. Heat may be generated externally with high-frequency waves targeting a tumor from a device outside the body. Internal heat may involve a sterile probe, including thin, heated wires or hollow tubes filled with warm water, implanted microwave antennae, or radiofrequency electrodes.

A patient's organ or a limb is heated for regional therapy, which is accomplished using devices that produce high energy, such as magnets. Alternatively, some of the patient's blood may be removed and heated before being perfused into an area that will be internally heated. Whole-body heating may also be implemented in cases where cancer has spread throughout the body. Warm-water blankets, hot wax, inductive coils, and thermal chambers may be used for this purpose.

### 6. Pancreatic Cancer Therapies

Exocrine. A key assessment that is made after diagnosis is whether surgical removal of the tumor is possible, as this is the only cure for this cancer. Whether or not surgical resection can be offered depends on how much the cancer has spread. The exact location of the tumor is also a significant factor, and CT can show how it relates to the major blood vessels passing close to the pancreas. The general health of the person must also be assessed, though age in itself is not an obstacle to surgery.

Chemotherapy and, to a lesser extent, radiotherapy are likely to be offered to most people, whether or not surgery is possible. Specialists advise that the management of pancreatic cancer should be in the hands of a multidisciplinary team including specialists A several aspects of oncology, and is, therefore, best conducted in larger centers.

Surgery with the intention of a cure is only possible in around one-fifth (20%) of new cases. Although CT scans help, in practice it can be difficult to determine whether the tumor can be fully resected, and it may only become apparent during surgery that it is not possible to successfully remove the tumor without damaging other vital tissues. Whether or not surgical resection can be offered depends on various factors, including the precise extent of local anatomical adjacency to, or involvement of, the venous or arterial blood vessels, as well as surgical expertise and a careful consideration of projected post-operative recovery. The age of the person is not in itself a reason not to operate, but their general performance status needs to be adequate for a major operation.

One particular feature that is evaluated is the encouraging presence, or discouraging absence, of a clear layer or plane of fat creating a barrier between the tumor and the vessels. Traditionally, an assessment is made of the tumor's proximity to major venous or arterial vessels, in terms of “abutment” (defined as the tumor touching no more than half a blood vessel's circumference without any fat to separate it), “encasement” (when the tumor encloses most of the vessel's circumference), or full vessel involvement. A resection that includes encased sections of blood vessels may be possible in some cases, particularly if preliminary neoadjuvant therapy is feasible, using chemotherapy and/or radiotherapy.

Even when the operation appears to have been successful, cancerous cells are often found around the margins of the removed tissue, when a pathologist examines them microscopically (this will always be done), indicating the cancer has not been entirely removed. Furthermore, cancer stem cells are usually not evident microscopically, and if they are present they may continue to develop and spread. An exploratory laparoscopy (a small, camera-guided surgical procedure) may therefore be performed to gain a clearer idea of the outcome of a full operation.

For cancers involving the head of the pancreas, the Whipple procedure is the most commonly attempted curative surgical treatment. This is a major operation which involves removing the pancreatic head and the curve of the duodenum together (“pancreato-duodenectomy”), making a bypass for food from the stomach to the jejunum (“gastro-jejunostomy”) and attaching a loop of jejunum to the cystic duct to drain bile (“cholecysto-jejunostomy”). It can be performed only if the person is likely to survive major surgery and if the cancer is localized without invading local structures or metastasizing. It can, therefore, be performed only in a minority of cases. Cancers of the tail of the pancreas can be resected using a procedure known as a distal pancreatectomy, which often also entails removal of the spleen. This can now often be done using minimally invasive surgery.

Although curative surgery no longer entails the very high death rates that occurred until the 1980s, a high proportion of people (about 30-45%) still have to be treated for a post-operative sickness that is not caused by the cancer itself. The most common complication of surgery is difficulty in emptying the stomach. Certain more limited surgical procedures may also be used to ease symptoms (see Palliative care): for instance, if the cancer is invading or compressing the duodenum or colon. In such cases, bypass surgery might overcome the obstruction and improve quality of life but is not intended as a cure.

After surgery, adjuvant chemotherapy with gemcitabine or 5-FU can be offered if the person is sufficiently fit, after a recovery period of one to two months. In people not suitable for curative surgery, chemotherapy may be used to extend life or improve its quality. Before surgery, neoadjuvant chemotherapy or chemoradiotherapy may be used in cases that are considered to be “borderline resectable” in order to reduce the cancer to a level where surgery could be beneficial. In other cases, neoadjuvant therapy remains controversial, because it delays surgery.

Gemcitabine was approved by the United States Food and Drug Administration (FDA) in 1997, after a clinical trial reported improvements in quality of life and a 5-week improvement in median survival duration in people with advanced pancreatic cancer. This was the first chemotherapy drug approved by the FDA primarily for a non-survival clinical trial endpoint. Chemotherapy using gemcitabine alone was the standard for about a decade, as a number of trials testing it in combination with other drugs failed to demonstrate significantly better outcomes. However, the combination of gemcitabine with erlotinib was found to increase survival modestly, and erlotinib was licensed by the FDA for use in pancreatic cancer in 2005.

The FOLFIRINOX chemotherapy regimen using four drugs was found more effective than gemcitabine, but with substantial side effects, and is thus only suitable for people with good performance status. This is also true of protein-bound paclitaxel (nab-paclitaxel), which was licensed by the FDA in 2013 for use with gemcitabine in pancreas cancer. By the end of 2013, both FOLFIRINOX and nab-paclitaxel with gemcitabine were regarded as good choices for those able to tolerate the side-effects, and gemcitabine remained an effective option for those who were not. A head-to-head trial between the two new options is awaited, and trials investigating other variations continue. However, the changes of the last few years have only increased survival times by a few months. Clinical trials are often conducted for novel adjuvant therapies.

The role of radiotherapy as an auxiliary (adjuvant) treatment after potentially curative surgery has been controversial since the 1980s. In the early 2000s the European Study Group for Pancreatic Cancer Research (ESPAC) showed prognostic superiority of adjuvant chemotherapy over chemoradiotherapy. The European Society for Medical Oncology recommends that adjuvant radiotherapy should only be used for people enrolled in clinical trials. However, there is a continuing tendency for clinicians in the US to be more ready to use adjuvant radiotherapy than those in Europe. Many clinical trials have tested a variety of treatment combinations since the 1980s but have failed to settle the matter conclusively.

Radiotherapy may form part of treatment to attempt to shrink a tumor to a resectable state, but its use on unresectable tumors remains controversial as there are conflicting results from clinical trials. The preliminary results of one trial, presented in 2013, “markedly reduced enthusiasm” for its use on locally advanced tumors.

PanNETs. Treatment of PanNETs, including the less common malignant types, may include a number of approaches. Some small tumors of less than 1 cm. that are identified incidentally, for example on a CT scan performed for other purposes, may be followed by watchful waiting. This depends on the assessed risk of surgery which is influenced by the site of the tumor and the presence of other medical problems. Tumors within the pancreas only (localized tumors), or with limited metastases, for example to the liver, may be removed by surgery. The type of surgery depends on the tumor location, and the degree of spread to lymph nodes.

For localized tumors, the surgical procedure may be much less extensive than the types of surgery used to treat pancreatic adenocarcinoma described above, but otherwise surgical procedures are similar to those for exocrine tumors. The range of possible outcomes varies greatly; some types have a very high survival rate after surgery while others have a poor outlook. As all this group are rare, guidelines emphasize that treatment should be undertaken in a specialized center. Use of liver transplantation may be considered in certain cases of liver metastasis.

For functioning tumors, the somatostatin analog class of medications, such as octreoide, can reduce the excessive production of hormones. Lanreotide can slow tumor growth. If the tumor is not amenable to surgical removal and is causing symptoms, targeted therapy with everolirnus or sunitinib can reduce symptoms and slow progression of the disease Standard cytotoxic chemotherapy is generally not very effective for PanNETs, but may be used when other drug treatments fail to prevent the disease from progressing, or in poorly differentiated PanNET cancers.

Radiation therapy is occasionally used if there is pain due to anatomic extension, such as metastasis to bone. Some PanNETs absorb specific peptides or hormones, and these PanNETs may respond to nuclear medicine therapy with radiolabeled peptides or hormones such as iobenguane (iodine-131-MIBC). Radiofrequency ablation (RFA), cryoablation, and hepatic artery embolization may also be used.

Palliative care. Palliative care is medical care which focuses on treatment of symptoms from serious illness, such as cancer, and improving quality of life. Because pancreatic adenocarcinoma is usually diagnosed after it has progressed to an advanced stage, palliative care as a treatment of symptoms is often the only treatment possible.

Palliative care focuses not on treating the underlying cancer, but on treating symptoms such as pain or nausea, and can assist in decision-making, including when or if hospice care will be beneficial. Pain can be managed with medications such as opioids or through procedural intervention, by a nerve block on the celiac plexus (CPB). This alters or, depending on the technique used, destroys the nerves that transmit pain from the abdomen. CPB is a safe and effective way to reduce the pain, which generally reduces the need to use opioid painkillers, which have significant negative side effects.

Other symptoms or complications that can be treated with palliative surgery are obstruction by the tumor of the intestines or bile ducts. For the latter, which occurs in well over half of cases, a small metal tube called a stet ray be inserted by endoscope to keep the ducts draining. Palliative care can also help treat depression that often comes with the diagnosis of pancreatic cancer.

Both surgery and advanced inoperable tumors often lead to digestive system disorders from a lack of the exocrine products of the pancreas (exocrine insufficiency). These can be treated by taking pancreatin which contains manufactured pancreatic enzymes, and is best taken with food. Difficulty in emptying the stomach (delayed gastric emptying) is common and can be a serious problem, involving hospitalization. Treatment may involve a variety of approaches, including draining the stomach by nasogastric aspiration and drugs called proton-pump inhibitors or H2 antagonists, which both reduce production of gastric acid. Medications like metoclopramide can also be used to clear stomach contents.

### VI. EXAMPLES

The following examples are included to demonstrate particular embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Pancreatic ductal adenocarcinoma (PDA) is one of the world's most lethal cancer with 5-10% five-year survival rate with no effective treatment available. PDA is characterized by immunosuppressive tumor microenvironment (TME) with high myeloid cell infiltration and low T cell responses. Retinoblastoma (Rb1) protein is a known tumor suppressor protein. In addition, there is accumulating evidence for its role in immune cell response, including regulation of apoptosis in myeloid cells.

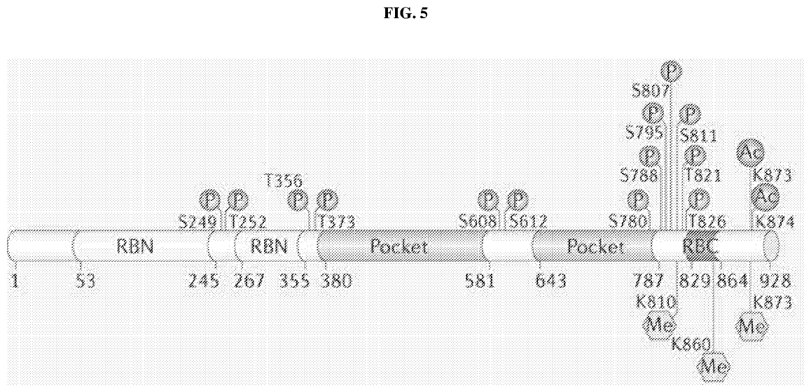

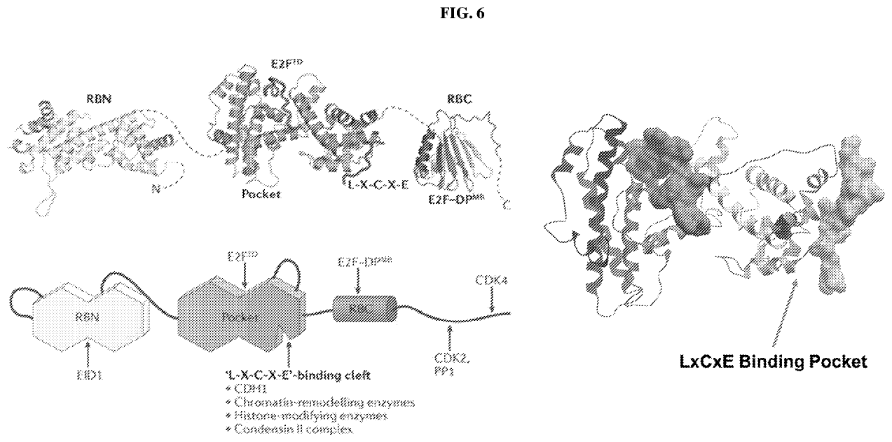

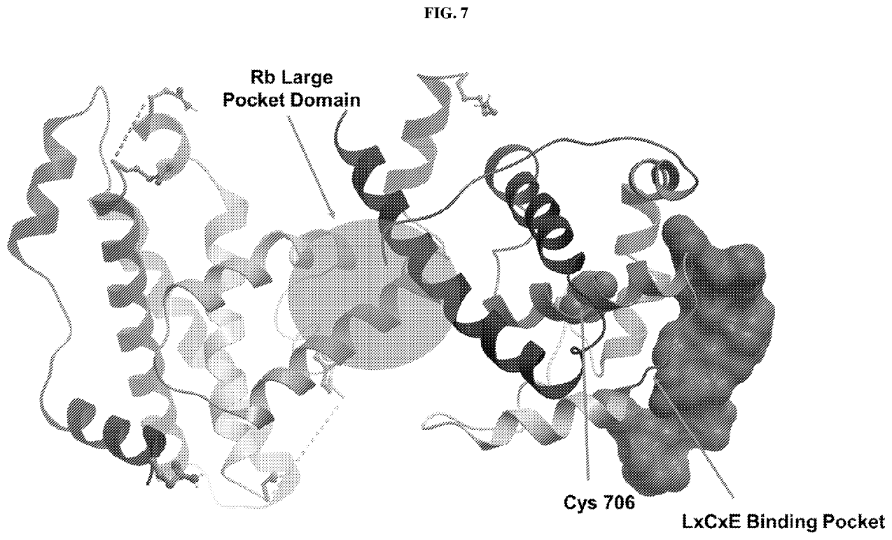

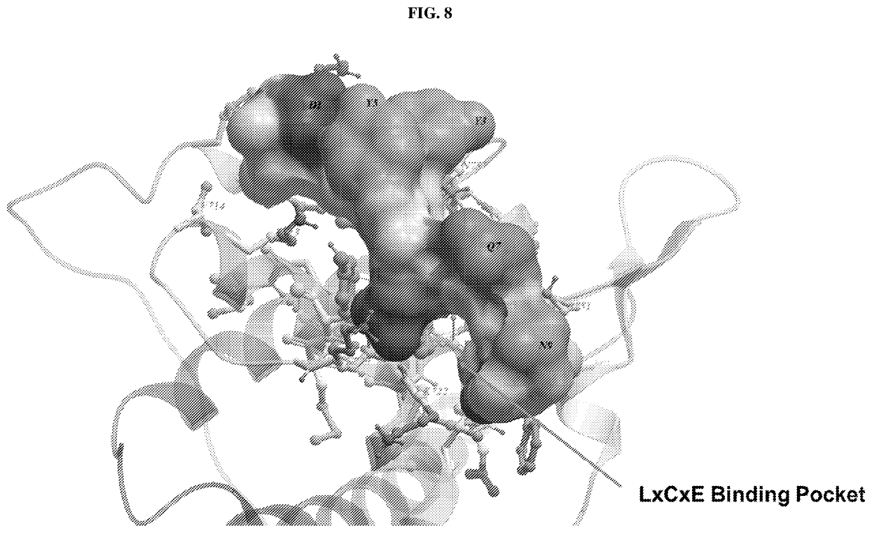

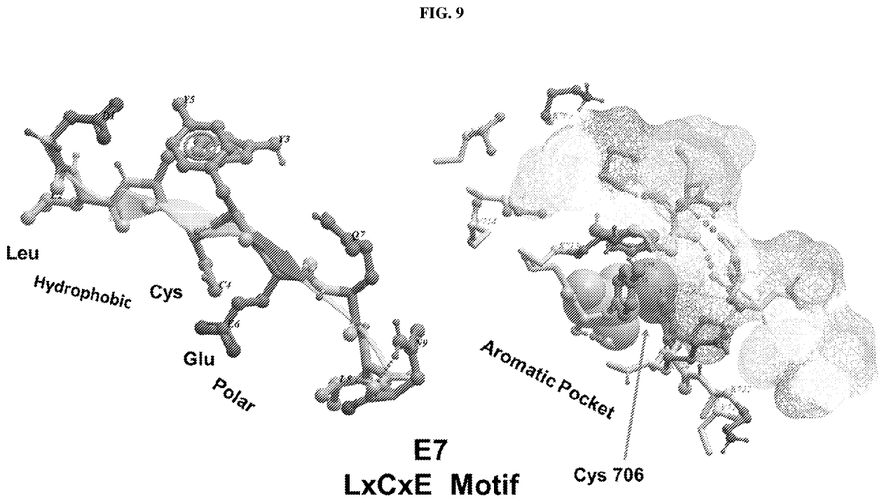

The Retinoblastoma protein is a master regulator of cell cycle progression and has 279 protein interactions. There are three major binding pockets (N-terminal, pocket, and C-terminal) with 14 phosphorylation sites. There are also 2 acetylation and 3 methylation sites in the C-terminus. See FIG. 5-9.

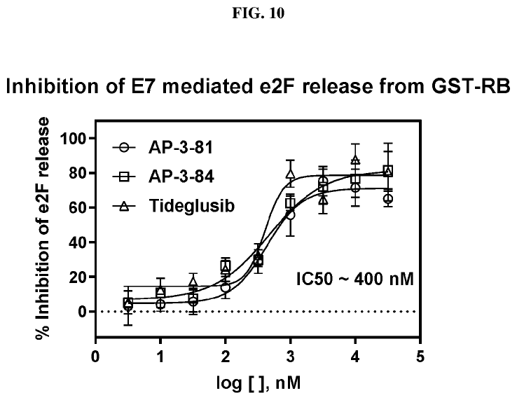

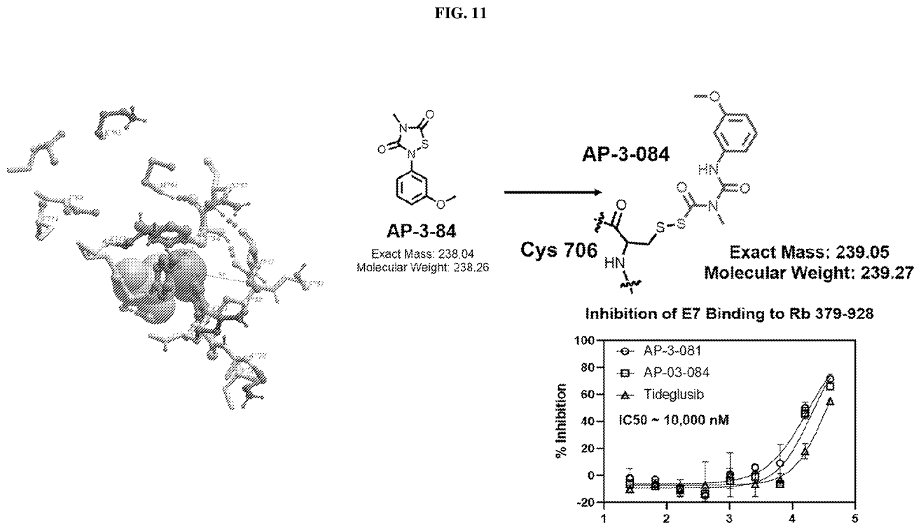

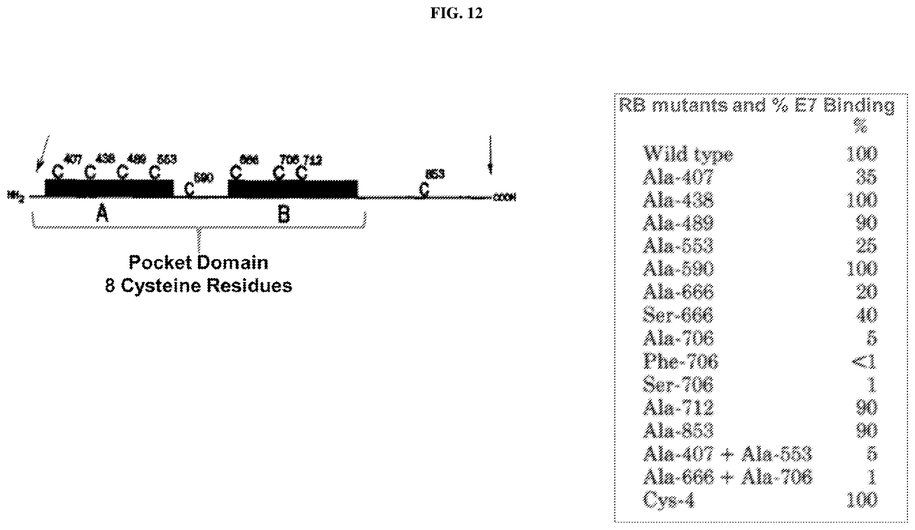

The inventors have developed the new small molecule AP-3-84 that is able to bind Rb1 and modulate its activity. AP-3-84 is believed to bind to the LxCxE binding pocket of the Rb1 protein. First, the compounds were tested for E7 mediated e2F1 release. Using an ELISA based assay for e2F release. Rb was bound to the assay plate and E2F1 was added. After washing with e2F1, the binding was detected using an E2F1 antibody. The HTRF assay was used to test for E2F release. GST-Rb and MBP-E2F1 are incubated together with an anti-MBP-Rb and an anti-GST-d2 HTRF antibodies. These result in a high signal. Then, the test compound and E2 were added so that when the e2F1 dissociated then the signal decreased. See FIG. 10. Without wishing to be bound by any theory, it is believed that the compounds such as AP-3-84 bind to the LxCxE binding pocket preventing E7 from binding. See the mechanistic study in FIG. 11. Previous studies by Merck and NCI showed that RB C706A mutation resulted in a RB protein that was defective in phosphorylation and oncoprotein binding. Mutation of Cys 706 resulted in loss of E7 binding. (Stirdivant et al., 1992; Kaye et al., 1990). Similarly, the binding of proteins is reduced with C706A mutants as well as C712A mutants. See FIG. 12. (Stirdivant et al., 1992).

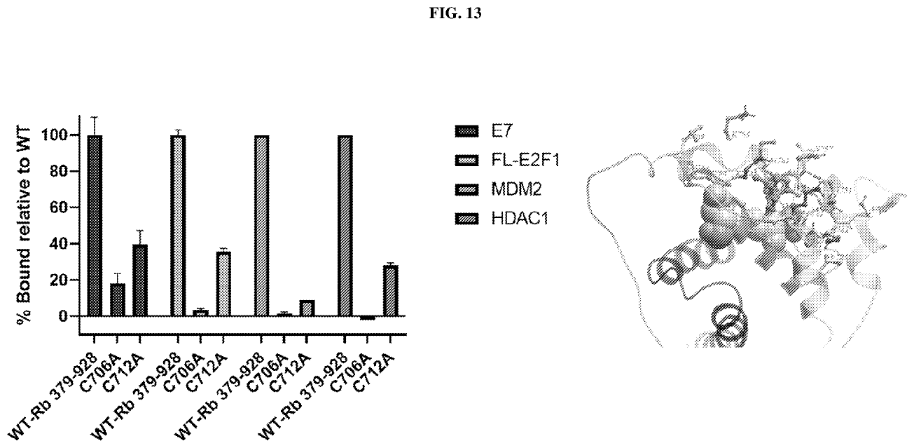

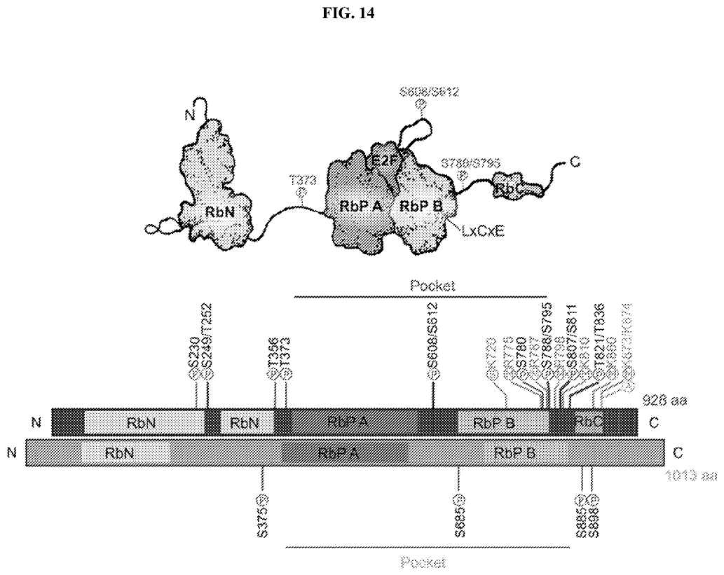

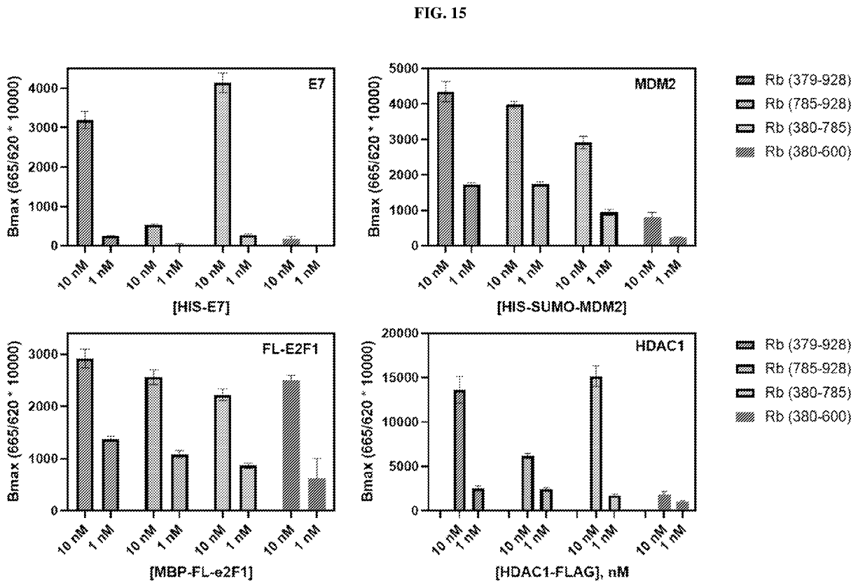

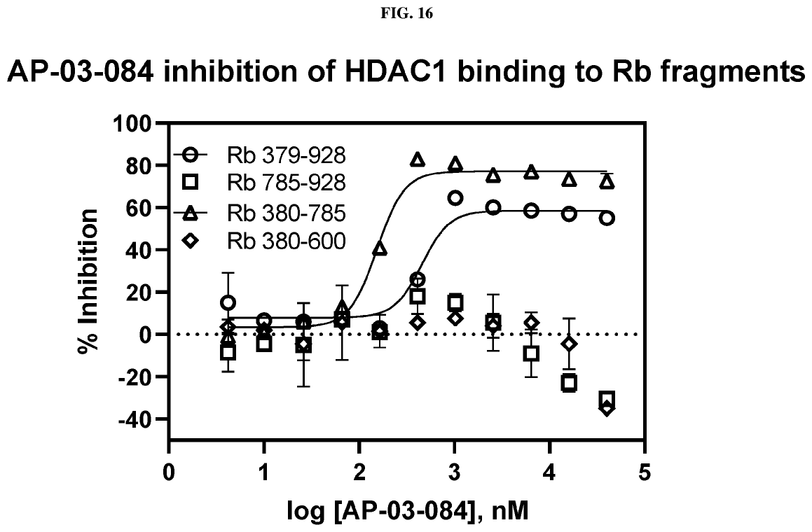

Additionally, the binding of different proteins was evaluated. The binding of E7, E2F1, MDM2, and HDAC1 were evaluated to Rb fragments. See FIGS. 13 & 14. The compounds tested herein where shown inhibit HDAC1 binding to the Rb fragments in the LxCxE binding pocket. See FIG. 15. The compounds were not able to inhibit HDAC1 binding in a Rb fragment that does not contain a LxCxE binding pocket. E2F and MDM binding were only partially inhibited. See FIG. 16.

Several compounds shown below were tested. The structures of these compounds are shown below.

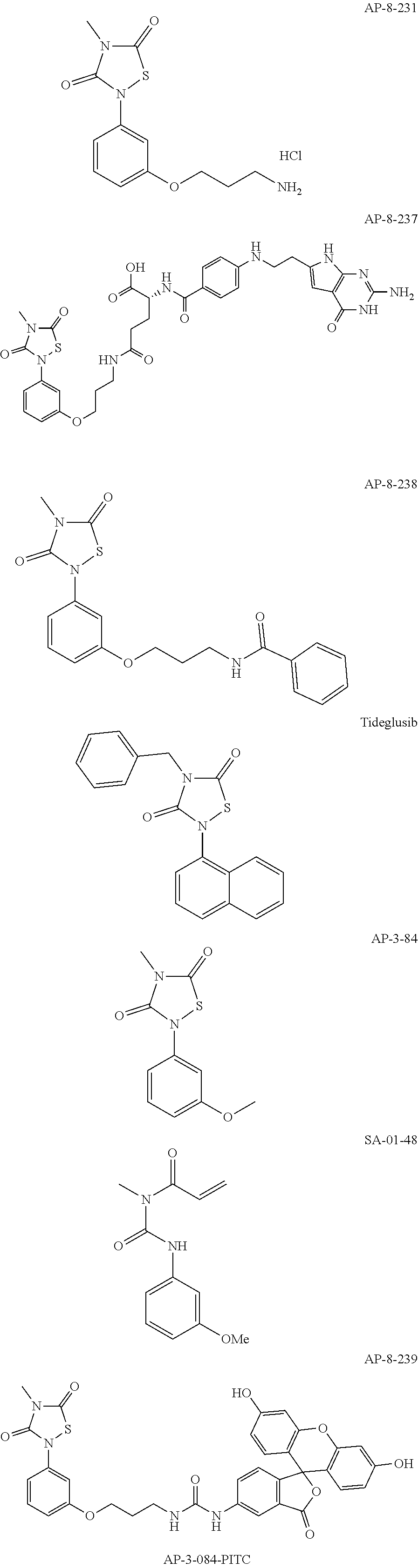

Table 1 below shows the inhibition values of the compound.

TABLE 1
Inhibition of Compounds

| | |
|---|---|
| Inhibition of | Inhibition of |
| AP-8-239 binding | HDAC1 binding |
| Rb-NT (380-785) | Rb-NT (380-785) |

| Compound | IC50, nM | Top | IC50, nM | Top |
|---|---|---|---|---|
| AP-08-231 | 362 | 83.3 | 77 | 72.0 |
| AP-08-237 | 22 | 85.9 | 171 | 72.0 |
| AP-08-238 | 54 | 90.6 | 83 | 71.0 |
| Tideglusib | 126 | 86.9 | 40 | 77.0 |
| AP-03-084 | 445 | 90.1 | 89 | 75.0 |
| SA-01-048 | 2923 | 83.8 | 12700 | 69.0 |

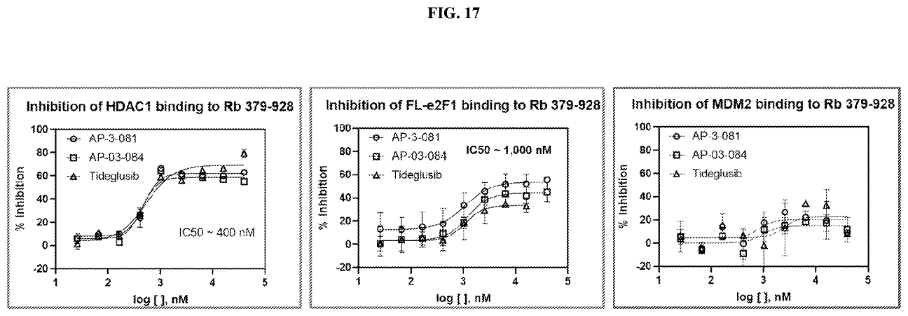

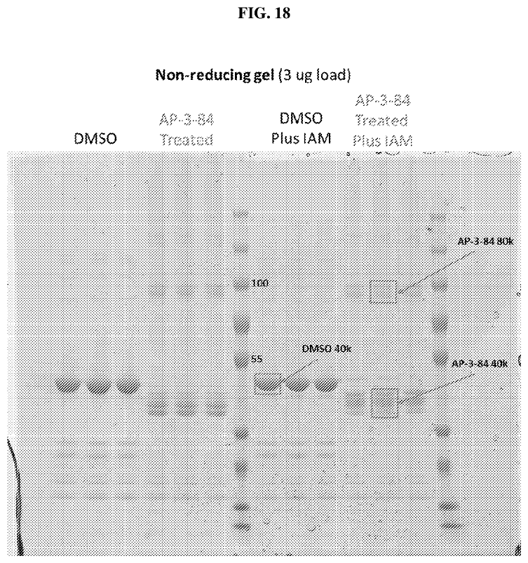

Further studies were conducted to evaluate the binding of the compounds to the Rb protein. Using MS, the compounds that had been treated with excess amounts of the compounds were then capped with iodoacetamide. The treatment resulted in cross-linking of the protein that resulted from disulfide linkages. See FIG. 17. Further evidence suggests that these compounds do not bind to these cysteine residues. Furthermore, there was a significant reduction in MS/MS sequencing events. These events suggest that Cys706 and Cys7l2 likely formed a disulfide bonds and that Cys438 may have formed a inter-protein disulfide bond. See FIG. 18. One explanation is that the compound activates Cys706 for nucleophilic attack from Cys712.

In order to confirm that, the Rb protein was treated with iodoacetamide to cap the Cys residues. The predicted chart is shown below.

| | | MALDI-MS | | |
|---|---|---|---|---|
| | Expected | observed | Wt | # of |
| Sample | mass (Da) | mass (Da) | difference | CH₂CONH₂ |
| RB + DMSO | 46,982 | 46,952 | | |
| RB + 84 | 47,220 | 46,993 | | |
| RB + 239 | 47,653 | 47,019 | | |
| RB + DMSO + | 47,439 | 47,419 | 467 | 8 |
| Iodoacetamide | | | | |
| RB + 84 + | 47,677 | 47,063 | 111 | 2 |
| Iodoacetamide | | | | |
| RB + 239 + | 48,110 | 47,320 | 368 | 6 |
| Iodoacetamide | | | | |

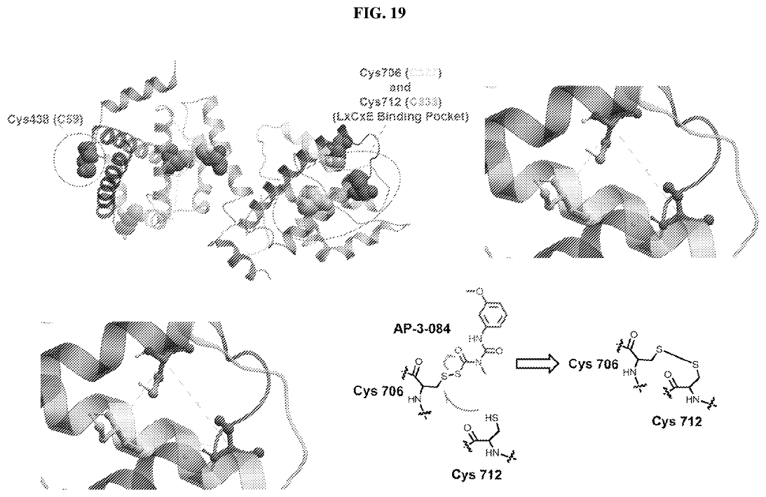

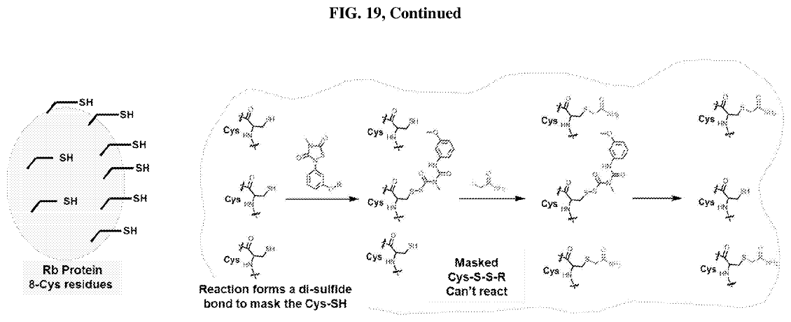

With the addition of the smaller 84 compound, 6 Cys residues were masked from reacting with the iodoacetamide while the larger 239 compound only blocked 2 Cys residues that resulted in improved selectivity. The proposed reaction is shown in FIG. 19.

While the compounds have been prepared, additional derivatives were prepared. The synthetic route is shown below.

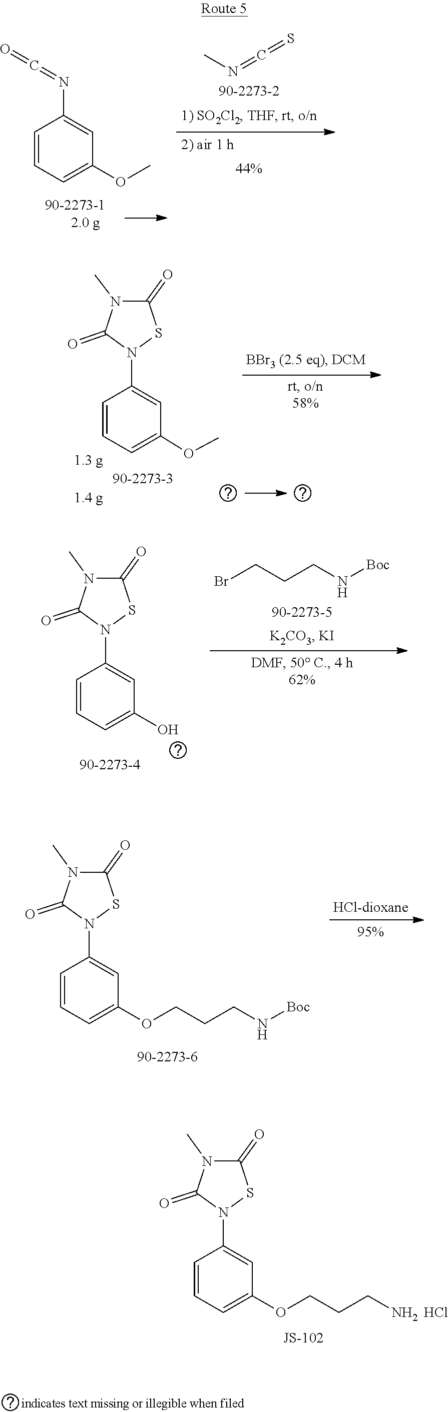

Originally, they found that this Rb1 modulator can induce cell death of TAMs and thioglycolate-induced macrophages, but not of the other cell types (neither tumor nor T cells). Gene expression changes upon AP-3-84 treatment showed the induction of oxidative and ER stress programs with a clear activation of p53-dependent genes and caspases.

The inventors tested the use of AP-3-84 as immunotherapy against PDA cancer growth using 2 PDA cell lines intrinsically characterized by low T cell and high T cell infiltration (T-low-PDA and T-high-PDA, respectively, originally generated and characterized by J. Li et al. (2018). In accordance with in vitro observations, AP-3-84 was able to reshape immune cell landscape in mice with T-high-PDA by depleting macrophages and inducing T cell infiltration. Importantly, those changes were accompanied by significant reduction of tumor growth. Addition of AP-3-84 to immunotherapy (anti-CD40/PD1/CTL4) was also able to decrease tumor burden in T-low-PDA model, which is otherwise resistant to this immunotherapy. Thus, AP-3-84 treatment was able to significantly improve the outcome of experimental PDA by altering immune cell subsets. Statistical analysis of the data was conducted using unpaired Student t-test criteria with additional false discovery rate adjustment for multiple comparisons for gene expression analysis.

In sum, targeting of Rb1 with the small molecule compound AP-3-84 induced cell death in tumor associated macrophages (TAMs). This depletion of TAMs resulted in a re-shaping of immune cell landscape and significant reduction in pancreatic tumor growth in mice upon AP-3-84 treatment. Based on these results, the inventors propose Rb1 modulation in myeloid cells as a promising target for PDA treatment.

Additionally, the inventors obtained test results showing the compounds to be effective in models of AML. AML cell lines (THP-1, AML-193, Kasumi-1) were cultured at different cell concentration (75000/ml, 125,000/ml and 200,000/ml) and exposed to 5, 10, 15, 20 μM concentration of AP-3-84. Cell differentiation was induced by 100 ng/ml PMA over 24 hours. Nonhemopoietic cancer cells (ID8, PDAC) were analyzed after exposure to AP-3-84. Cell death outcomes were measured by Annexin-V using flowcytometry or viability measures by Alamar blue assay. Protein levels were measured by Western blots at intervals between 30 min and 6 hours after exposure to AP-3-84 for total Rb1, phosphorylated Rb1, ATF3, BIM, NOXA, BaK, Bax, cleaved PARP, cleaved Caspase-3. Cytoplasmic and mitochondrial lysates were examined. Confocal immunocytochemistry on Rb1 expression in nucleus versus cytoplasm was performed.

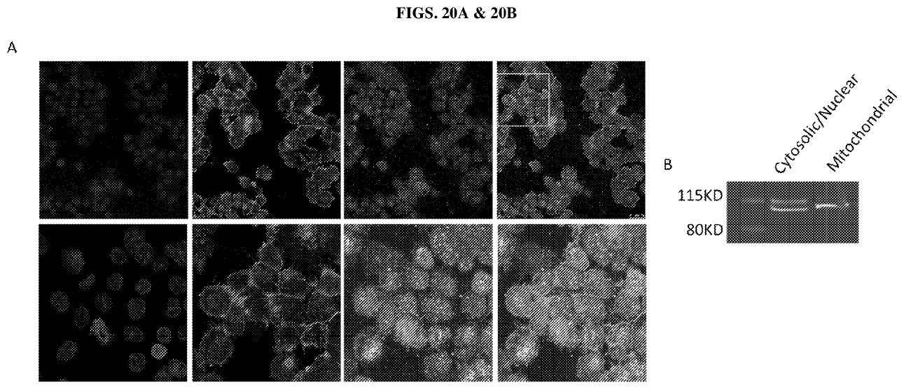

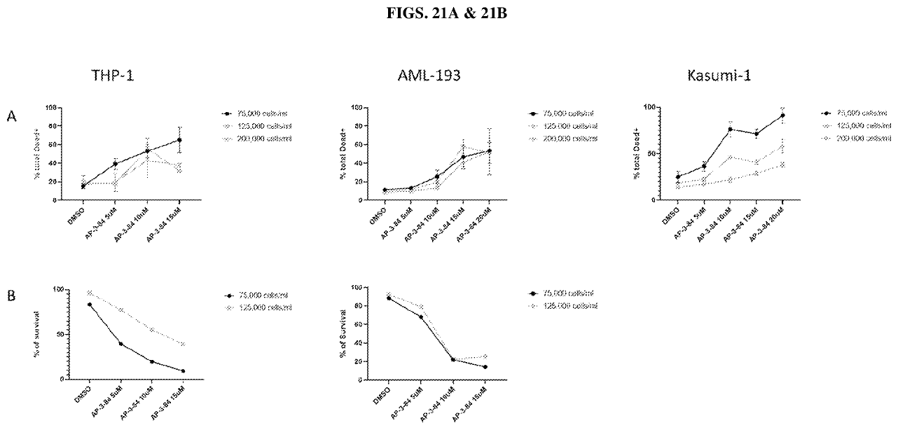

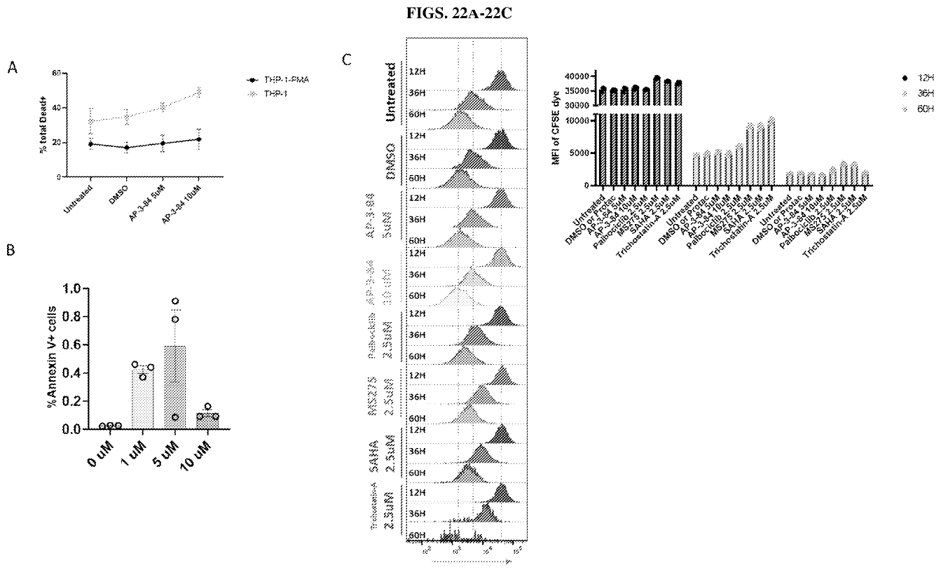

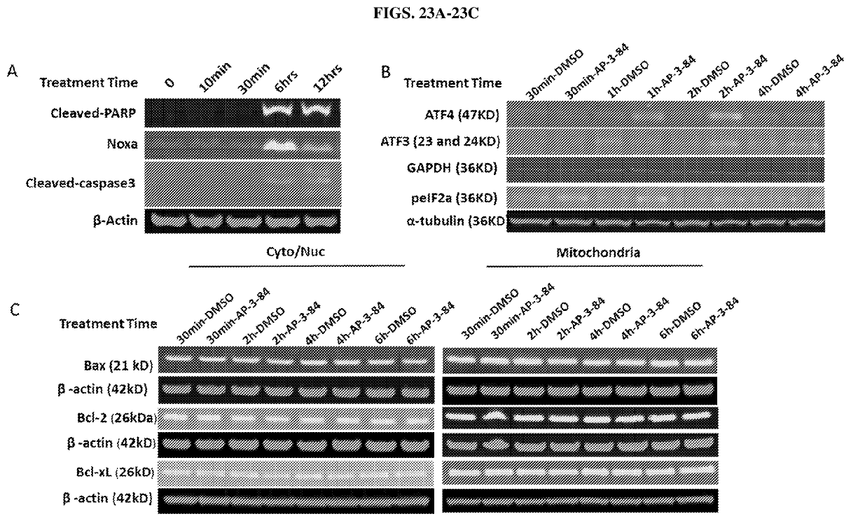

Rb1 protein expression was documented by immunocytochemistry in nucleus and cytoplasm of THP1 cells consistent with its detection in nuclear and mitochondrial lysates (Western). AP-3-84 induction of cell death was observed at 10 μM or higher concentration, with a 5 minute exposure sufficient to cause cell death in 70-80% of cells 24 hours later. Cell death by AP-3-84 was specific to dividing AML cells as no cell death was induced in PMA differentiated THP1 cells or nonhemopoietic cancer cells. No effect in cell division rate by AP-3-84 was present in nonhemopoitic cancer cells indicating a lack of cell cycle regulation. Mechanistically, AP-3-84 induced higher levels of ER-stress signals (higher ATF4, ATF3) and increased mitochondrial protein levels of NOXA, BIM, cleaved-PARP, and cleaved Caspase-3. Bax and Bak levels were unchanged after AP-3-84 exposure. See FIGS. 20-23.

All of the compounds, formulations, and methods disclosed and claimed herein can be made and executed without undue experimentation in light of the present disclosure. While the compounds, formulations, and methods of this disclosure have been described in terms of preferred embodiments, it will be apparent to those of skill in the art that variations may be applied to the compounds, formulations, and methods, as well as in the steps or in the sequence of steps of the method described herein without departing from the concept, spirit, and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VII. REFERENCES

The following references to the extent that they provide exemplary procedural or other details supplementary to those set forth herein, are specifically incorporated herein by reference.
Handbook of Pharmaceutical Salts: Properties, and Use, Stahl and Wermuth Eds.), Verlag Helvetica Chimica Acta, 2002.
Jensen et al., BMC Med. Imaging, 8:16, 2008
March's Advanced Organic Chemistry: Reactions, Mechanisms, and Structure, 2007.
Saghafi et al., Neurosci. Lett., 488:247-251, 2011.
WO 2014/089067
Li et al., Immunity 49(1):178-193, 2018
Stgirdivant et al., J. Biol. Chem., 267:14846-51, 1992.
Kaye et al., Proc. Natl. Acad. Sci. USA, 87:6922-26, 1990.

1. A method of treating cancer comprising administering to a patient in need thereof a therapeutically effective amount of a compound of the formula: 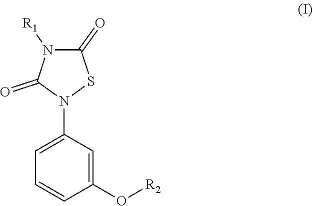
wherein:
R₁ is hydrogen, alkyl_{(C≤12)}, substituted alkyl_{(C≤12)}, aralkyl_{(C≤12)}, or substituted aralkyl_{(C≤12)}; and
R₂ is an organic moiety; or
a compound of the formula: 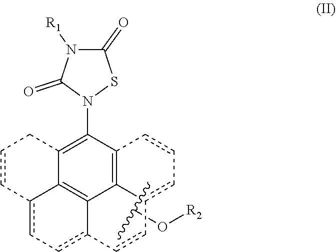
wherein:
the dashed lines represent one or more fused aromatic ring systems;
R₁ is hydrogen, alkyl_{(C≤12)}, substituted alkyl_{(C≤12)}, aralkyl_{(C≤12)}, or substituted aralkyl_{(C≤12)}; and
R₂ is an organic moiety; or
a compound of the formula: 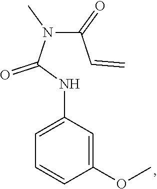 [SMILES: C=CC(=O)N(C)C(=O)Nc1cccc(OC)c1]
or a pharmaceutically acceptable salt or tautomer thereof.

2. The method of claim 1, wherein R₁ is alkyl_{(C≤12)} or substituted alkyl_{(C≤12)}.

3. The method of claim 2, wherein R₁ is alkyl_{(C≤12)}.

4. The method of claim 1, wherein R₁ is methyl.

5. The method of claim 2, wherein R₁ is substituted alkyl_{(C≤12)}.

6. The method of claim 1, wherein R₁ is aralkyl_{(C≤12)} or substituted aralkyl_{(C≤12)}.

7. The method of claim 6, wherein R₁ is aralkyl_{(C≤12)}.

8. The method of claim 7, wherein R₁ is benzyl.

9. The method of claim 1, wherein R₂ is an organic moiety selected from a linking group selected from —C(O)—, —C(S)—, —C(O)O—, —C(S)O—, —OC(O)—, —OC(S)—, —C(O)NR_{b}—, —C(S)NR_{b}—, —NR_{b}C(O)—, —NR_{b}C(S)—, —OC(O)O—, —OC(S)O—, —NR_{b}C(O)NR_{b}—, —NR_{b}C(S)NR_{b}—, —S(O)ₓ—, —OS(O)ₓO—, —OS(O)ₓ—, —S(O)ₓO—, and a combination thereof, wherein: x is 0, 1, or 2; and R_{b} is hydrogen, alkyl_{(C≤6)}, or substituted alkyl_{(C≤6)}, a small molecule targeting agent, hydrogen, amino, cyano, halo, hydroxy, nitro, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, alkylamino_{(C≤12)}, dialkylamino_{(C≤12)}, cycloalkylamino_{(C≤12)}, cycloalkyl(alkyl)amino_{(C≤12)}, dicycloalkylamino_{(C≤12)}, alkoxy_{(C≤12)}, aryloxy_{(C≤12)}, acyloxy_{(C≤12)}, acyl_{(C≤12)}, amido_{(C≤12)}, alkylsulfonyl_{(C≤12)}, arylsulfonyl_{(C≤12)}, or a substituted version thereof, or a combination thereof.

10. The method of claim 9, wherein R₂ is alkyl_{(C≤12)} or substituted alkyl_{(C≤12)}.

11. The method of claim 10, wherein R₂ is alkyl_{(C≤12)}.

12. The method of claim 11, wherein R₂ is methyl.

13. The method of claim 10, wherein R₂ is substituted alkyl_{(C≤12)}.

14. The method of claim 13, wherein R₂ is aminopropyl.

15. The method of claim 14, wherein R₂ is 3-aminopropyl.

16. The method of claim 14, wherein R₂ is a combination of alkyl_{(C≤12)} or substituted alkyl_{(C≤12)} and amido_{(C≤12)} or substituted amido_{(C≤12)}.

17. The method of claim 16, wherein R₂ is a combination of alkyl_{(C≤12)} and amido_{(C≤12)}.

18. The method of claim 17, wherein the amido_{(C≤12)} of R₂ is —C(O)Ph.

19. The method according to claim 1, wherein the alkyl_{(C≤12)} of R₂ is ethylene or propylene.

20. The method of claim 19, wherein the alkyl_{(C≤12)} of R₂ is propylene.

21. The method of claim 1, wherein R₂ is a combination of an alkyl_{(C≤12)} or substituted alkyl_{(C≤12)}, a linking group, and an imaging agent.

22. The method of claim 21, wherein the alkyl_{(C≤12)} of R₂ is ethylene or propylene.

23. The method of claim 22, wherein the linking group of R₂ is —C(O)—, —C(S)—, —C(O)O—, —C(S)O—, —OC(O)—, —OC(S)—, —C(O)NR_{b}—, —C(S)NR_{b}—, —NR_{b}C(O)—, —NR_{b}C(S)—, —OC(O)O—, —OC(S)O—, —NR_{b}C(O)NR_{b}—, —NR_{b}C(S)NR_{b}—, —S(O)ₓ—, —OS(O)ₓO—, —OS(O)ₓ—, —S(O)ₓO—, and a combination thereof, wherein: x is 0, 1, or 2; and R_{b} is hydrogen, alkyl_{(C≤6)}, or substituted alkyl_{(C≤6)}.

24. The method of claim 23, wherein the linking group of R₂ is —OC(S)O— or —NR_{b}C(S)NR_{b}—.

25. The method of claim 24, wherein the linking group of R₂ is —NR_{b}C(S)NR_{b}—.

26. The method of claim 21, wherein the imaging agent is a fluorophore.

27. The method of claim 26, wherein the fluorophore is fluorescein.

28. The method of claim 1, wherein R₂ is a combination of one or more alkyl_{(C≤12)} or substituted alkyl_{(C≤12)}, a targeting group, an aryl_{(C≤12)} or substituted aryl_{(C≤12)}, and an amino acid.

29. The method of claim 28, wherein the alkyl_{(C≤12)} of R₂ is ethylene or propylene.

30. The method of claim 28, wherein the targeting group of R₂ is a nucleotide or nucleotide analog.

31. The method of claim 28, wherein the aryl_{(C≤12)} of R₂ is a benzenediyl.

32. The method of claim 1, wherein the compound is of formula II.

33. The method of claim 32, wherein the compound comprises two aryl rings.

34. The method of claim 32, wherein the compound is further defined as: 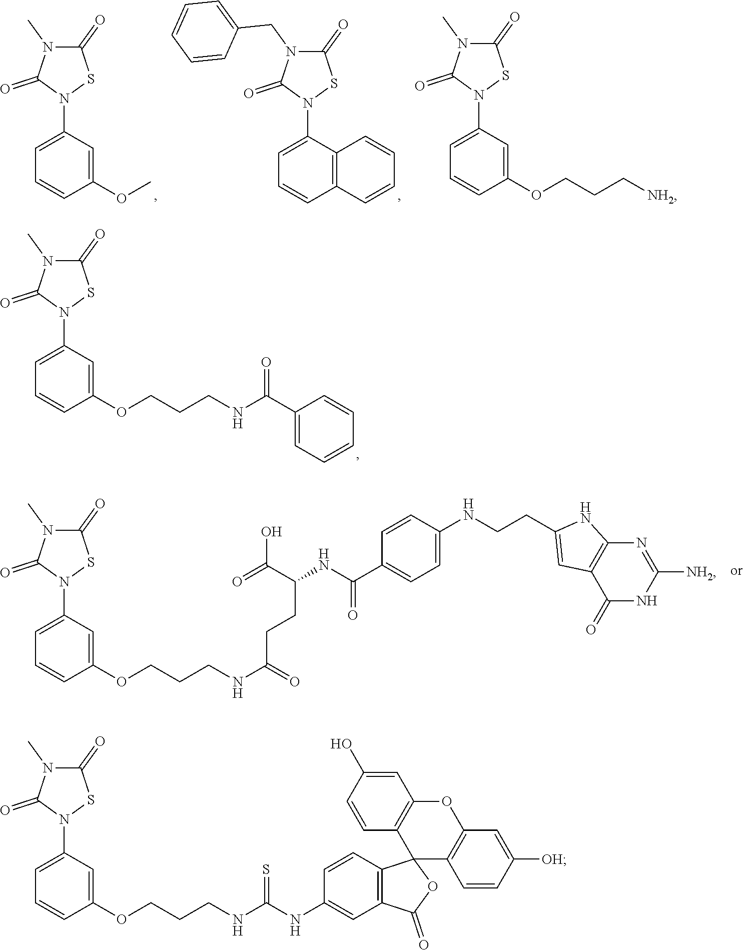
or a pharmaceutically acceptable salt thereof.

35. The method of claim 1, wherein the cancer is a solid tumor cancer.

36. The method of claim 1, wherein the cancer is a carcinoma, sarcoma, lymphoma, leukemia, melanoma, mesothelioma, multiple myeloma, or seminoma.

37. The method of claim 1, wherein the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid.

38. The method of claim 37, wherein the cancer is a cancer of the pancreas.

39. The method of claim 38, wherein the cancer is an adenocarcinoma of the pancreas.

40. The method of claim 39, wherein the pancreatic adenocarcinoma is a pancreatic ductal adenocarcinoma.

41. The method of claim 1, wherein the cancer is a leukemia.

42. The method of claim 41, wherein the leukemia is acute myeloid leukemia.

43. The method of claim 1, wherein the method comprises modulating the myeloid cells of the cancer.

44. The method of claim 1, wherein the cancer is an operable cancer.

45. The method of claim 1, wherein the cancer is inoperable.

46. The method of claim 45, wherein the inoperable cancer becomes operable after treatment with the compound.

47. The method of claim 1, wherein the cancer is a drug resistant cancer.

48. The method of claim 1, wherein the cancer recurrent and/or metastatic.

49. The method of claim 1, wherein the method comprises injecting the compound directly into the tumor.

50. The method of claim 49, wherein the compound is formulated as an injectable solution.

51. The method of claim 1, wherein the method comprises administering the compound systemically.

52. The method of claim 51, wherein the compound is formulated for oral or intravenous administration.

53. The method of claim 1, wherein the method further comprises alleviating pain.

54. The method of claim 1, wherein the method further comprises administering a second therapeutic regimen to said patient.

55. The method of claim 54, wherein the second therapeutic regimen is surgery, radiotherapy, immunotherapy, genetic therapy, or a second chemotherapeutic compound.

56. The method of claim 55, wherein the second therapeutic regimen comprises gemcitabine and/or paclitaxel.

57. The method of claim 1, further comprising at least a second, third, fourth, fifth, sixth, seventh, eighth, ninth or tenth administration of said compound.

58. A method of reducing the size of a tumor comprising administering to a patient in need thereof a therapeutically effective amount of a compound of the formula: 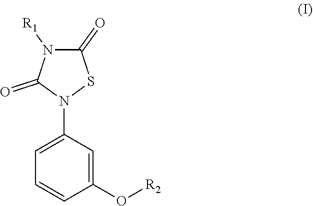
wherein:
R₁ is hydrogen, alkyl_{(C≤12)}, substituted alkyl_{(C≤12)}, aralkyl_{(C≤12)}, or substituted aralkyl_{(C≤12)}; and
R₂ is an organic moiety;
or a pharmaceutically acceptable salt or tautomer thereof.

59. A compound of the formula: 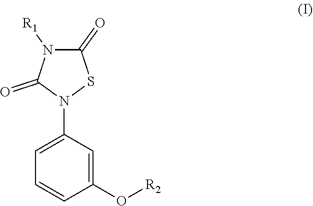
wherein:
R₁ is hydrogen, alkyl_{(C≤12)}, substituted alkyl_{(C≤12)}, aralkyl_{(C≤12)}, or substituted aralkyl_{(C≤12)}; and
R₂ is an organic moiety;
provided the compound is not: 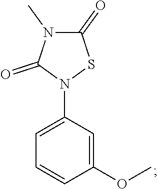 [SMILES: COc1cccc(-n2sc(=O)n(C)c2=O)c1]
or a pharmaceutically acceptable salt thereof.

60. The compound of claim 59, wherein R₁ is alkyl_{(C≤12)} or substituted alkyl_{(C≤12)}.

61. The compound of claim 59, wherein R₁ is methyl.

62. The compound of claim 59, wherein R₂ is an organic moiety selected from a linking group selected from —C(O)—, —C(S)—, —C(O)O—, —C(S)O—, —OC(O)—, —OC(S)—, —C(O)NR_{b}—, —C(S)NR_{b}—, —NR_{b}C(O)—, —NR_{b}C(S)—, —OC(O)O—, —OC(S)O—, —NR_{b}C(O)NR_{b}—, —NR_{b}C(S)NR_{b}—, —S(O)ₓ—, —OS(O)ₓO—, —OS(O)ₓ—, —S(O)ₓO—, and a combination thereof; wherein: x is 0, 1, or 2; and R_{b} is hydrogen, alkyl_{(C≤6)}, or substituted alkyl_{(C≤6)}, a small molecule targeting agent, hydrogen, amino, cyano, halo, hydroxy, nitro, or alkyl_{(C≤12)}, cycloalkyl_{(C≤12)}, alkenyl_{(C≤12)}, alkynyl_{(C≤12)}, aryl_{(C≤12)}, aralkyl_{(C≤12)}, heteroaryl_{(C≤12)}, heteroaralkyl_{(C≤12)}, heterocycloalkyl_{(C≤12)}, alkylamino_{(C≤12)}, dialkylamino_{(C≤12)}, cycloalkylamino_{(C≤12)}, cycloalkyl(alkyl)amino_{(C≤12)}, dicycloalkylamino_{(C≤12)}, alkoxy_{(C≤12)}, aryloxy_{(C≤12)}, acyloxy_{(C≤12)}, acyl_{(C≤12)}, amido_{(C≤12)}, alkylsulfonyl_{(C≤12)}, arylsulfonyl_{(C≤12)}, or a substituted version thereof, or a combination thereof.

63. The compound of claim 59, wherein R₂ is alkyl_{(C≤12)}, substituted alkyl_{(C≤12)}, amido_{(C≤12)}, substituted amido_{(C≤12)}, or a combination thereof.

64. The compound of claim 59, wherein R₂ is substituted alkyl_{(C≤12)}.

65. The compound of claim 59, wherein R₂ is a combination of alkyl_{(C≤12)} and amido_{(C≤12)}.

66. The compound of claim 59, wherein R₂ is —CH₂CH₂CH₂NHC(O)Ph.

67. The compound of claim 66, wherein the compound is further defined as: 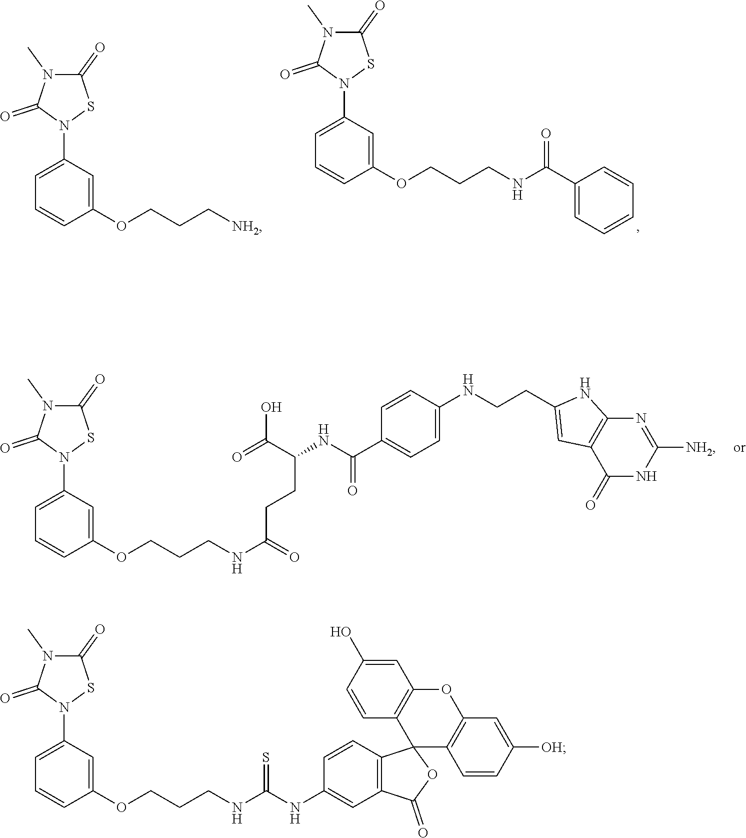 [SMILES: Cn1c(=O)sn(-c2cccc(OCCCN)c2)c1=O ; Cn1c(=O)sn(-c2cccc(OCCCNC(=O)c3ccccc3)c2)c1=O ; Cn1c(=O)sn(-c2cccc(OCCCNC(=O)CC[C@@H](NC(=O)c3ccc(NCCc4cc5c(=O)[nH]c(N)nc5[nH]4)cc3)C(=O)O)c2)c1=O ; Cn1c(=O)sn(-c2cccc(OCCCNC(=S)Nc3ccc4c(c3)C(=O)OC43c4ccc(O)cc4Oc4cc(O)ccc43)c2)c1=O]
or a pharmaceutically acceptable salt thereof.

68. A pharmaceutical composition comprising a compound of claim 59 and a pharmaceutically acceptable excipient.
